(19) United States
(12) Patent Application Publication
(10) Pub. No.: US 2004/0136866 A1
(43) Pub. Date: Jul. 15, 2004

(54) Planar nanowire based sensor elements, devices, systems and methods for using and making same

(75) Inventors: George Pontis, Redwood City, CA (US); Walter Stonas, Campbell, CA (US); Calvin Chow, Portola Valley, CA (US); J. Wallace Parce, Palo Alto, CA (US); Yaoling Pan, Union City, CA (US); Linda T. Romano, Sunnyvale, CA (US); Shahriar Mostarshed, San Mateo, CA (US)

Correspondence Address: QUINE INTELLECTUAL PROPERTY LAW GROUP, P.C., P O BOX 458, ALAMEDA, CA, 94501, US

(73) Assignee: NANOSYS, Inc.

(21) Appl. No.: 10/608,775

(22) Filed: Jun. 26, 2003

Related U.S. Application Data
(60) Provisional application No. 60/392,205, filed on Jun. 27, 2002.
(60) Provisional application No. 60/426,076, filed on Nov. 13, 2002.

(51) Int. Cl. G01N 33/00

(52) U.S. Cl. 422/57

(57) ABSTRACT

Sensor elements, and devices that incorporate those elements are provided, where the sensor comprises a thin semiconductor nanowire that is disposed upon, and integral to, an insulating substrate, where the nanowire is electrically coupled to a sensing surface that binds or otherwise interacts with an analyte of interest to yield a measurable effect on the nanowire element. The methods utilize extremely thin semiconductor on insulator substrates to provide nanowire dimensions for sensing elements that are fabricated using conventional semiconductor fabrication processes.

### CROSS-REFERENCE TO RELATED APPLICATIONS

This application is related to Provisional U.S. Patent Application No. 60/392,205, filed Jun. 27, 2002, the full disclosure of which is hereby incorporated herein by reference in its entirety for all purposes.

### BACKGROUND OF THE INVENTION

Analytical operations, and particularly bioanalytical operations have been subject to major advancements over the past thirty years, including the advent of powerful genetic analysis and manipulation techniques, e.g., the polymerase chain reaction and recombinant DNA technology, powerful analytical techniques and chemistries, e.g., rapid sequencing methods, fluorescent indicators, and microfluidic analysis systems.

Despite all of the advances made in these arenas, there still exists a need for greater sensitivity, higher accuracy, greater reproducibility, and more miniaturized, integrated and automated approaches to many analyses.

Researchers have developed chemical sensing technologies that employ chemically sensitive field effect transistors, or ChemFETs, to detect the presence and/or concentration of different chemical and biochemical species. These ChemFET sensors typically employ integrated circuit fabrication technology in the production of sensor elements, and provide data in the form of electrical signals, as opposed to the optical signals that had been conventionally used. In brief, the interaction of a charged analyte with a moiety associated with a conductive sensor element carries with it a field effect that modulates the electrical properties of the sensor element, e.g., the conductance. This conductance can then be monitored and used as an indication of the level of interaction, which can, in turn, be used to extrapolate the concentration of the analyte. The use of non-optical detection provides a sensitive means for detecting interactions while alleviating the need for special labeling chemistries for each different type of interaction of interest, which labeling schemes can be expensive, toxic, potentially interfering of low sensitivity, and in many cases, simply unavailable.

Such sensors, while providing numerous advantages over conventional analytical methods, suffer from issues of cost, sensitivity, and manufacturability. Advances in sensitivity have been gained by using extremely small scale sensing elements, e.g., carbon nanotubes and semiconductor nanowires, and that has allowed for proportionally greater effects from small numbers of interaction events, e.g., binding events (See, Cui, et al., Science 293, 1289-1292 (2001)). The small size of these “nanostructures” allows for substantially increased sensitivity, since the field effect of a bound analyte affects a greater portion of the sensor element than the larger sensors that had been previously described. Specifically, the field effect of a bound analyte modulates the conductance across a greater percentage of the cross section of the nanowire or nanotube, and thus more substantially affects its measurable conductance.

Again, despite increases in sensitivity, nanotubes and nanowires also suffer from certain disadvantages, primarily relating to their manufacturability and integratability. Briefly, both silicon nanotubes and nanowires are generally fabricated as discrete individual elements, e.g., individual wires or tubes. The individual wires are then positioned to bridge two electrical contacts or electrodes and then coated with a binding agent, either directly or through an intermediate surface coating, layer or treatment, to provide specificity for an analyte of interest. Binding of a complementary analyte to the binding agent yields a change in the electrical environment and thus the electrical properties of the wire between the electrodes, which is monitored by detecting a shift in the conductance of the wire. While their small size provides increased sensitivity for nanowire based sensors, difficulties in positioning such small scale structures, results in higher cost manufacturing processes as a result of complex manufacturing processes and substantially low process yields. Simply put, it is difficult to accurately deposit nanostructures in desired locations on substrates. This difficulty increases as one seeks to integrate the sensor element with other more complex electrical elements. In particular, the integration of nanowire structures with other, potentially more complex structures and systems in a practical fashion, necessarily presupposes the ability to accurately place nanostructures within a given device.

Accordingly, there exists a need to be able to duplicate the sensitivity of the nanowire based sensor, while at the same time improving the manufacturability and usability of the sensor and devices that integrate other elements with the sensor. The present invention provides a simple, elegant means for meeting these and a variety of other important needs.

### BRIEF SUMMARY OF THE INVENTION

The present invention provides sensor elements, and devices that incorporate those elements, where the sensor includes a thin semiconductor nanowire that is disposed upon an insulating substrate, where the nanowire is electrically coupled to a sensing surface that binds or otherwise interacts with an analyte of interest. The methods of the invention utilize extremely thin semiconductor on insulator substrates to provide nanowire dimensions for sensing elements that are fabricated using more conventional semiconductor fabrication processes.

Thus, in some aspects, the invention comprises a nanosensor which comprises: a semiconductor element (having length and width dimensions parallel to the insulating substrate and a depth dimension orthogonal to the insulating substrate (the depth dimension being less than 500 nm)) integral to an insulating substrate, and a sensing surface, comprising at least a first functional moiety that is capable of interacting with a first analyte of interest, electrically coupled to the semiconductor element.

In some embodiments, the depth dimension of the semiconductor element is less than 200 nm, less than 100 nm, less than 50 nm, less 25 nm, or is between about 15 nm and about 100 nm. In other embodiments, the semiconductor element of the nanosensor comprises silicon and the insulator comprises silicon dioxide. Furthermore, the functional moiety of the nanosensor can comprise, e.g., a biochemical, a metal, a metal oxide, etc. In some embodiments, the nanosensor can include functional moieties that comprise one member of a paired group, e.g., one member of: a receptor:ligand pair, a binding protein:ligand pair, an antibody:epitope pair, an antibody fragment:epitope pair, a pair of complementary oligonucleotides, or a phosphorylated protein:multivalent metal ion pair.

The sensing surfaces of the nanosensors herein can optionally have the functional moiety coupled directly to a surface of the semiconductor element. Other embodiments can have the functional moiety directly coupled to the surface of the semiconductor element via a linker molecule. The sensing surface of the nanosensors herein can also comprise the first functional moiety associated with a layer disposed over the semiconductor element. Also, the layer disposed over the semiconductor element can comprises an insulator layer, a metal layer (e.g., gold, platinum, tin, a metal oxide layer), etc. Also, in some embodiments herein, the nanosensors can include semiconductor elements which have first and second segments which each can comprise different doping.

The nanosensors herein can also further comprise an electrical circuit that is electrically coupled to the semiconductor element. Such electrical circuit can comprise, e.g., a buffering circuit, a multiplexing circuit (e.g., that is electrically coupled to at least one additional semiconductor element), or an amplification circuit. Such additional semiconductor elements can be integral to an insulating substrate, have length and width dimensions parallel to the insulating substrate, have a depth dimension (less than 100 nm) orthogonal to the insulating substrate, and a sensing surface (comprising a second functional moiety for interacting with a second analyte of interest) electrically coupled to the semiconductor element. In some embodiments, such second functional moiety is different from the first functional moiety. The nanosensors herein, can further comprise first and second electrical contacts that are electrically coupled to different points along the length dimension of the semiconductor element.

In yet other aspects, the invention comprises a nanosensor which includes: a semiconductor element having a longitudinal axis, and which is attached to an insulating substrate such that the longitudinal axis is parallel to the insulating substrate, and also wherein the semiconductor element comprises a depth dimension (that is less than 500 nm) orthogonal to the substrate. Additionally, such nanosensors include first and second electrical contacts in electrical communication with the semiconductor element at first and second different points along the longitudinal axis, respectively, as well as, a sensing surface electrically coupled to the semiconductor element, which has at least a first functional moiety immobilized thereon, so that interaction of an analyte of interest with the functional moiety induces a change in an electrical property of the semiconductor element.

In other aspects, the invention comprises an array which, in turn, comprises a first nanosensor element (having a first semiconductor element integral to an insulating substrate and having length and width dimensions parallel to the insulating substrate, and a depth dimension orthogonal to the insulating substrate (the depth dimension being less than 500 nm), as well as a first sensing surface (comprising at least a first functional moiety for interacting with a first analyte of interest) electrically coupled to the semiconductor element), and, at least a second nanosensor element (comprising a second semiconductor element integral to an insulating substrate, and having length and width dimensions parallel to the insulating substrate, and a depth dimension orthogonal to the insulating substrate (the depth dimension being less than 500 nm) and a sensing surface (the second sensing surface comprising at least a second functional moiety for interacting with a second analyte of interest) electrically coupled to the second semiconductor element).

In some such embodiments, such arrays include depth dimensions of the first and second semiconductor elements that are less than 200 nm or less than 100 nm. In yet other embodiments, the arrays of the invention include first and second nanosensor elements that are independently electrically addressable; are disposed in a single fluid reservoir; or are each electrically coupled to a multiplexing circuit. In other embodiments, the first and second functional moieties and/or the first and second analytes of interest are different. In yet other embodiments, the first and second analytes of interest are the same analyte. Furthermore, the first and second nanosensor elements can be disposed in different fluid reservoirs.

In yet other aspects, the invention comprises a method of fabricating a nanosensor, through providing a semiconductor layer on an insulating substrate (wherein the semiconductor layer is less than 500 nm thick) defining an elongated structure from the semiconductor layer (the structure having length and width dimensions that are parallel to the insulating substrate and a depth dimension orthogonal to the insulating substrate that is less than 500 nm), and providing a sensing surface (comprising a functional moiety that interacts with an analyte of interest to induce a change in an electrical property of the elongated structure) electrically coupled to the elongated structure.

In some such embodiments, the providing of the semiconductor layer on an insulating substrate comprises providing a semiconductor layer that is less than 200 nm thick or is less than 100 nm thick. In some embodiments of the method, the depth dimension is substantially equal to the thickness. Furthermore, the semiconductor layer on an insulating substrate can comprise a semiconductor on insulator substrate (e.g., a silicon on insulator (SOI) substrate such as a silicon layer on a silicon dioxide layer). The semiconductor on an insulator substrate can also comprise a SiMOX wafer. In some embodiments of such methods, the step of defining can comprise: coating the semiconductor layer with a resist; exposing and developing the resist to produce a pattern in the resist that corresponds to the structure to be defined; protecting the pattern that corresponds to the structure to be defined; and, removing the semiconductor layer that does not correspond to the structure to be defined, thereby defining the structure. Such methods can also include wherein the exposing step comprises irradiating defined portions of the resist with an electron beam or irradiating defined portions of the resist with light.

In further aspects, the invention comprises an analytical system which includes: a nanosensor (which comprises: a semiconductor element integral to an insulating substrate, and which has length and width dimensions parallel to the insulating substrate and a depth dimension orthogonal to the insulating substrate (the depth dimension being less than 500 nm) and a ratio of the length dimension to the depth dimension being greater than 500); and a sensing surface that is electrically coupled to the semiconductor element, the sensing surface comprising a functional moiety capable of interacting with an analyte of interest; and, a detector electrically coupled to the nanosensor for measuring conductance of the semiconductor element. In some such embodiments, the depth dimension of the semiconductor element is less than 200 nm or is less than 100 nm. In yet other embodiments, the system can further comprise a fluid containing vessel (e.g., a fluidic conduit, a microfluidic channel, a well in a multiwell plate, etc.) with the sensing surface of the nanosensor being at least partially disposed within such fluid vessel. In yet other embodiments, the system can further comprise a computer operably coupled to the detector, the computer being operably programmed to receive and store conductance data from the detector or a fluid handling system fluidly connected to the nanosensor for directing fluid samples into contact with the sensing surface of the nanosensor.

In still other aspects, the invention comprises a method of analyzing a sample material, such method comprising: providing a nanosensor (having a semiconductor element integral to an insulating substrate, and having length and width dimensions parallel to the insulating substrate, and a depth dimension orthogonal to the insulating substrate, the depth dimension being less than 500 nm); and, a sensing surface electrically coupled to the semiconductor element, the sensing surface comprising a functional moiety capable of interacting with an analyte of interest; and, contacting a sample material with the sensing surface of the nanosensor; and determining a concentration of the analyte of interest in the sample material. In such embodiments, the depth dimension of the semiconductor element can be less than 200 nm or less than 100 nm. Additionally, the step of determining in such methods can comprise measuring a conductance of the semiconductor element, and correlating the conductance to a concentration of the analyte of interest. Furthermore, the step of contacting in such methods can comprise immersing the sensing surface in the sample material or flowing the sample material over the sensing surface.

These and other objects and features of the invention will become more fully apparent when the following detailed description is read in conjunction with the accompanying figures.

### BRIEF DESCRIPTION OF THE FIGURES

FIG. 1A schematically illustrates a system incorporating a nanowire based sensor. FIG. 1B schematically illustrates the operation of the nanowire based sensor portion of the system shown in FIG. 1A.

FIG. 2 schematically illustrates a planar nanowire based sensor of the invention and schematically illustrates its operation.

FIG. 3 schematically illustrates a typical fabrication process used in making the nanowire sensor elements of the invention.

FIG. 4 schematically illustrates an alternate fabrication process to that shown in FIG. 3.

FIG. 5 schematically illustrates FIB fabrication of nanowire elements in planar, e.g., SOI substrates.

FIG. 6 illustrates a further alternate process for fabricating planar nanowire elements using spacer technology.

FIG. 7 schematically illustrates a nanowire sensor element having at least a first level of integrated elements fabricated along with it. In FIG. 7A, enlarged ohmic contact regions are illustrated as being provided in the base substrate, while FIG. 7B schematically illustrates inclusion of ‘fan out’ connections, as well as enlarged ohmic contact regions, for producing higher density arrays of interfaceable nanowire sensor elements.

FIG. 8 schematically illustrates an array of nanosensor elements in accordance with the present invention, which array is also integrated into a fluidic or microfluidic conduit system.

FIG. 9 schematically illustrates an overall analytical system that comprises a sensor of the present invention in conjunction with fluid handling systems for delivering materials to the sensor and processing or computing capabilities to control operation of the sensor, and receive, store and analyze data from the sensor.

FIGS. 10A and 10B schematically illustrates sensor devices of the present invention that incorporate backside ohmic contacts for a nanowire element.

FIGS. 11A and 11B schematically illustrate the use of a metallic sensing surface that is either disposed over the nanowire element (FIG. 11A) or separate from the nanowire element but still electrically coupled thereto (FIG. 11B).

FIG. 12 schematically illustrates the use of a metal sensing surface that is substantially structurally decoupled from, but electrically coupled to, the nanowire element of the overall sensor device.

FIGS. 13A-C show plotted sensor data from a planar nanowire sensor device of the invention.

FIGS. 14A-C show sensor data from an APTES modified planar nanowire sensor device of the invention.

FIG. 15 shows plotted data tracking leakage to a metal trace through a polymeric coating.

FIGS. 16A-F schematically illustrate the use of recessed or set-back metallic plates and sacrificial layers.

### DETAILED DESCRIPTION OF THE INVENTION

I. Nanoscale Sensors, Generally

The present invention generally provides novel sensors and sensing systems, particularly chemical and biochemical sensing systems, devices, elements, methods of fabricating such sensors and systems, and methods that employ such nanoscale sensing and/or detection elements. Such sensing systems, devices and methods are useful in a broad range of applications, including medical diagnostics, environmental testing, biological research, process monitoring, and the like.

The sensors described herein have the advantages of previously described nanoscale sensors, such as sensitivity, while providing numerous additional advantages, as well as overcoming many of the problems of the previously described sensors. In particular, the sensing devices of the invention will generally include integrated circuitry ancillary to the sensing element, such as protection circuits, e.g., resistors, diodes, and the like, to protect the relatively delicate sensing elements, multiplexing circuits for connecting to multiple sensing elements in a single device, interfacing components, e.g., contact pads or zones, to facilitate interfacing with external electrical systems, e.g., directly or through pin outs, etc., and potentially a variety of other circuit elements.

In addition, the production of these sensing elements employs fabrication methods and materials that overcome many of the difficulties that plague other nanoscale sensing technologies, e.g., carbon nanotubes, etc., such as difficulties in positioning, integration and the like. The methods of fabricating the nanoscale sensors, as described herein, are expected to provide substantially improved process yields and quality. Equally as important, these methods also are expected to improve integratability of these sensors, while providing comparable or greater sensitivity relative to previously described nanosensors.

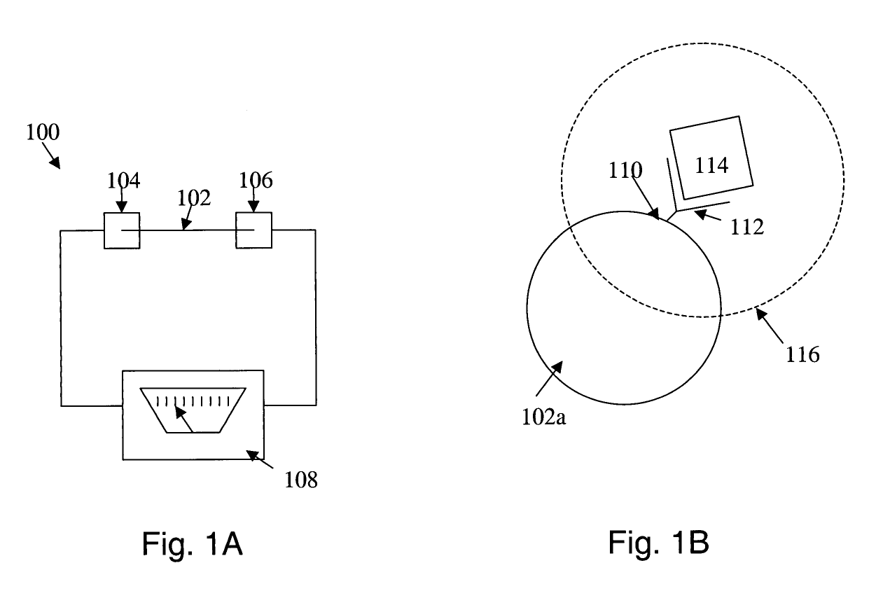

As noted previously, nanoscale chemical sensors typically derive their improved sensitivity from the extremely small scale structures that define them, and from the relative effects that a single or few binding events can have on those structures. Operation of a simple nanowire based sensor is schematically illustrated in FIGS. 1A and 1B. As shown in FIG. 1A, a simple system 100 includes a nanowire element 102 that is positioned bridging two electrical contacts, e.g., a current source and a drain, 104 and 106. These two contacts are, in turn connected to a system for measuring changes in conductance between the two contacts, e.g., an ohm meter 108. FIG. 1B schematically illustrates a close up cross sectional view of a previously described type of nanowire element 102a during an analytical operation. Briefly, the nanowire 102a is electrically coupled to an active or sensing surface 110 that, as shown, includes a chemical group that is capable of interacting with an analyte of interest, e.g., functional moiety 112. When functional moiety 112 interacts with an analyte of interest, e.g., analyte 114, having a localized charge density relative to the supporting solution, e.g., positive or negative, the field effect from this charge density (as indicated by dashed line 116) penetrates a portion of the nanowire 102a, modulating the conductance of at least a portion of the nanowire 102a.

Because this effect is across a relatively large portion of the cross-section of the wire, it produces a substantial and readily measurable shift in the overall conductance, either increasing or decreasing, of the nanowire. Thus, as used herein, the phrase “capable of interacting with an analyte of interest” generally refers to any interaction that will bring or maintain the analyte of interest in sufficient proximity to the sensing surface, and for sufficient duration, that such interaction will yield a measurable change in the properties, i.e., electrical properties like conductance, of the nanowire element of the devices described herein. In particularly preferred aspects, such interactions involve binding or coupling reactions, whether covalent or noncovalent, e.g., van der Walls forces, hydrophobic/hydrophilic interactions etc. Typically, a reference electrode is also provided in the solution which is being analyzed (not shown) to control the potential of the bulk solution relative to the sensor element, to provide a baseline level of solution potential against which the device conductivity is measured.

As noted previously, one of the difficulties associated with nanowire sensors is their manufacturability. In particular, both solid semiconductor nanowires and carbon nanotubes are generally produced in bulk, as collections of discrete nanoscale structures. In the case of solid semiconductor nanowires, for example, the wires are epitaxially grown from colloidal precursors disposed on planar substrates by chemical vapor deposition (CVD) techniques (See, e.g., Gudiksen et al, J. Am. Chem. Soc. 122, 8801-8802 (2000) and Cui et al., App. Phys. Lett. 78, 2214-2216 (2001)). The resulting wire elements are then harvested or separated from the substrate to yield a collection of discrete, individual, free standing nanowires. Nanotubes, while fabricated from a different process (see, e.g., Published International Patent Application No. WO 00/09443) yield the same type of collection of discrete, individual elements. In order to then use one or more nanowires or nanotubes in an overall sensor system, one must position the wire element in the operative portion of the sensing device, i.e., between two electrical contacts and within the sensing region, e.g., a fluid reservoir. Because of their extremely small size, such positioning often relies upon random or semi random placement methods, e.g., flowing solutions of nanotubes or wires over derivatized substrates and hoping for binding of the wire in a correct position and orientation. While these and other methods have been used to successfully perform the positioning operation, the yields of these methods have not approached the levels that would be necessary to provide a commercially reasonable manufacturing process.

In addition, as systems employing nanowire based sensors become more complex, they will require even greater levels of integration, e.g., coupling one or more nanowires with more complex circuits and systems. Commercially practicable fabrication of systems that have any integration beyond just the baseline operability level effectively requires accurate and repeatable positioning of the nanoscale elements. Similarly, commercial practicability also will require the ability to produce devices and systems with consistently high quality and sensitivity. This level of consistency may be difficult to acquire with techniques currently employed in nanowire-based device production, e.g., in growth and positioning.

II. Planar Nanowire Based Sensors of the Invention

The present invention generally employs more conventional semiconductor fabrication technology in producing nanoscale sensor elements in a planar, substrate based process, rather than producing free-standing bulk elements that must then be assembled. In doing so, the methods described herein take advantage of the built in integration of these conventional fabrication processes, as well as of the substantial pool of available technical expertise, equipment and high quality, commercially available starting materials.

In general, the sensors or sensor elements of the present invention include thin semiconductive paths or “nanowires” that are not free-standing elements, but are, instead, disposed upon an insulating substrate. As used herein, the term “nanowire” generally refers to a conductive or semiconductive element that has at least one cross-sectional dimension that is less than 1 μm, e.g., in the nanoscale range, preferably about 500 nm or less, and more preferably, 100 nm or less. Such nanowires typically will have an aspect ratio of the smallest cross-sectional dimension to its longest dimension of greater than 10, preferably, greater than 100, and in many cases, greater than 500 or even 1000.

The sensor elements of the invention are typically fabricated from thin semiconductor layers that are predisposed upon and integral to insulator substrates. In preferred aspects, silicon on insulator (SOI) substrates or substrates that employ other semiconductors (e.g., other Group IV semiconductors, or Group III-V and II-VI semiconductors) on insulator layers are preferred as they are readily commercially available with semiconductor layers that have high quality crystal structures that range in thickness down to less than 100 nm, and can be as thin as about 250 angstroms, 150 angstroms or less. As used herein, the cross sectional dimension of the nanowire element that is typically variable is the width of the nanowire element. As discussed herein, the width dimension of a nanowire element refers to the shorter dimension parallel to the insulator layer in the nanoscale wire element, while the length dimension refers to the longer dimension parallel to the insulator. The thickness or depth dimension refers to the dimension that is orthogonal to the insulator substrate.

Fabricating these nanowires from such thin semiconductor on insulator substrates allows one to take advantage of the very small cross-sectional dimensions (thickness), purity and high quality crystal structure of the semiconductor layer that such commercially available substrates offer, without having to provide for this dimension during the process of fabricating the nanowire element, as is the case for free standing nanowires. Further, because the thickness of these semiconductor layers is defined by the starting material, the process of defining a nanowire that is as sensitive as the previously described nanowire sensors, takes place in only two dimensions, length and width, and can define the nanowire in place, e.g., in the desired position of the completed device. This process avoids problems associated with inconsistencies in cross-section in the fabrication of free-standing nanowires, as well as difficulties in positioning them.

The process of defining the nanowire then simply involves the use of conventional high resolution lithographic techniques, e.g., e-beam lithography, or high resolution photolithography, to define the length and width of the wire in the semiconductor layer, with the thickness being predetermined. Alternatively, for certain applications, the semiconductor layer is sufficiently thin that conventional photolithographic methods can be used to define the length and width dimensions of the sensor element, while still providing sensor elements with desired sensitivity.

Thin layer nanowire based sensors are believed to possess all of the advantages of the previously described nanowire sensors, e.g., sensitivity, while avoiding the greatest disadvantages of those previously described sensors, e.g., manufacturability and integration. Specifically, the small thickness dimension of the thin layer nanowire sensors of the invention allows for sensitivity of previously described nanowire sensors, while the process used in fabricating such sensors allows those sensors to be fabricated in place, e.g., prepositioned.

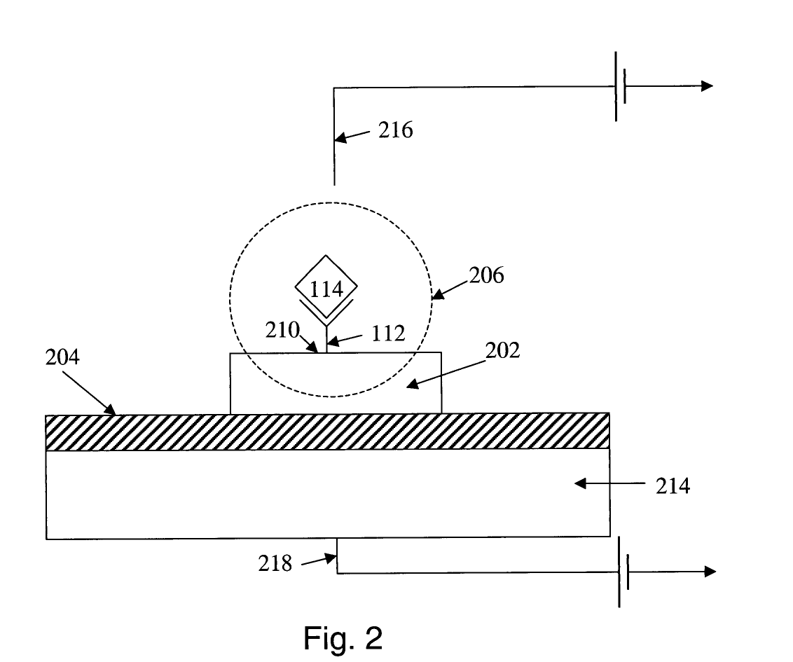

FIG. 2 schematically illustrates the operation of a sensor element of the present invention when employed in a system similar to that shown in FIG. 1A. As shown, the sensor element or “nanowire” 202, illustrated in cross section (which corresponds to sensor 102 in the system illustrated in FIG. 1A), is defined from a thin semiconductor layer disposed on an insulating substrate 204 (that is, in turn, disposed on a bulk semiconductor substrate 214. The nanowire 202 is provided electrically coupled to a sensing surface 210. As shown, the sensing surface comprises the exposed surface of the nanowire 202. The sensing surface 210 includes a functional moiety 112 that is capable of interacting with an analyte of interest. When an analyte of interest, 114, that includes a localized charge density relative to its fluid environment, interacts with the functional moiety on the sensing surface 210, the interaction induces a shift in the conductance of the nanowire 202 as a result of the field effect (as shown by dashed line 206) of analyte 114.

With a thin layer nanowire 202 having an appropriate thickness, this effect can be as substantial, if not more substantial than that achieved with a typical nanowire, e.g., wire 102a as schematically illustrated in FIG. 1B. Furthermore, because these thin layer wires are fabricated from commercially available semiconductor wafers in two dimensions, they are readily fabricated using conventional semiconductor processing techniques, e.g., photo or e-beam lithography and etching, e.g., reactive ion etching (RIE), using conventional processing equipment. The use of conventional semiconductor fabrication processes further enables facile integration of the nanoscale sensor element with other components of the system on the same substrate, from simple contact regions to more complex circuitry including diodes, resistors, transistors, amplifiers, multiplexers, and the like, whether for integration of more complex functionality on a single device or for protection of the sensor circuit from transient electrical impulses, e.g., static discharge, etc.

FIG. 2 also shows the inclusion of an optional reference electrode 216 that is placed into contact with the fluid to be analyzed, in order to control the potential of the solution relative to the sensor element. Also shown is a back gate contact 218 that applies a potential to the underlying semiconductor substrate 214, in order to bias the nanowire component into inversion, depletion or accumulation regimes. In particular, one can adjust the sensitivity of nanowire sensors, whether p or n doped, by biasing the sensor element appropriately.

In using conventional semiconductor fabrication techniques to produce sensor elements “in situ,” e.g., prepositioned in a desired location on a supporting substrate and/or with interfacing elements or other integrated elements, one can substantially simplify the process of producing nanowire based sensor elements. This simplification produces a substantial increase in process yields over the previously described low yield processes that required separate fabrication of the nanowire elements and difficult, low yield positioning steps. In particular, the sensor elements are effectively prepositioned, eliminating this particularly difficult step and its consequent yield losses. Additionally, it permits the inclusion of protective circuit elements from the initial formation of the sensor elements to prevent losses incurred during the manufacturing process from transient electrical impulses. Additionally, by employing high quality wafers, e.g., having monocrystalline semiconductor layers, one can substantially improve the quality of sensor elements that are produced, again, improving the yields of the overall process and reducing manufacturing costs. Finally, conventional semiconductor processing equipment and expertise is widely available, allowing facile integration of the fabrication processes of the present invention into existing fabrication facilities.

As noted above, high resolution lithography can be employed in defining the length and width dimensions of the nanoscale elements of the invention well into the nanoscale range, e.g., down to 10 or 20 to 50 nm widths. For those applications where single molecule resolution is desired, e.g., to detect a single interaction or binding event, it will typically be desirable to provide sensing elements having length or width dimensions on this order. In particular, it will generally be advantageous in such applications to provide sensing elements whose electrical properties can be substantially impacted upon interaction with, e.g., binding to, a single molecule. As such, for these applications, while the depth dimension, as defined by the starting substrate material is typically less than 100 nm, the width dimension will typically be less than 500 nm, preferably, less than 200 nm and in many cases, less than 100 nm, and down to 20 or even 10 nm, although process yields would be expected to drop as the lower limits of fabrication are approached.

For a number of applications, however, such sensitivity may not be required. For example, in a number of applications, analyte molecules will not be limiting in the sample material that is being analyzed. In such cases, it is expected that increasing the width of the sensing element while maintaining its depth at the dimensions provided above, will reduce single molecule sensitivity. However, increases in the size of the sensing surface with its consequent increase in the number of analyte interaction or binding sites is expected to offset such decreases in sensitivity for samples that are not analyte limited. Thus, while the impact on the properties of the nanosensor of a single binding event is less, there is a greater likelihood for more binding events, which should yield a similar, or the same impact. In such cases, the sensor elements of the invention would provide advantages of previously described microscale or macroscale sensors, e.g., large sensing surface areas to maximize binding interactions, with advantages of nanowire based sensors, e.g., sensitivity to smaller numbers of binding events. Thus, the ability to continuously scale the width of the nanoscale element for a wide variety of different applications, e.g., from 10 or 20 nm up to 10 μm or larger, while not sacrificing overall sensitivity, provides additional advantages of the present planar nanowire sensors over previously described nanowire based sensors where cross-sectional dimensions, e.g., diameters, are generally uniformly small, e.g., in the sub 100 nm range.

In additional aspects, one may increase the effective sensing surface of the sensor element without increasing the overall footprint of the sensing surface portion of the device. In particular, one may fabricate the sensing surface as a non flat surface, e.g., as a series of ridges and troughs or corrugations, in order to increase the available surface for sensing interactions, without increasing the overall footprint of the sensing surface portion of the sensor device. Typically, such sensing surfaces may be fabricated in such a fashion by readily available microfabrication techniques, e.g., photolithographic etching of troughs or wells in the sensing surface (which may then be coated with metal, oxide, nitride or other appropriate surfaces, or used directly as the sensing surface, as described elsewhere herein.

In addition to the advantages of ease of fabrication and integration described herein, planar processed nanowire elements also possess flexibility in terms of device structure and composition that are either lacking or more difficult in free standing nanowire structures. In particular, using conventional semiconductor processing technologies, one can readily vary the structure and/or composition of a planar nanowire element along its length. Typically, such modifications in free standing nanowires are either impossible, in terms of certain structural modifications, or require fundamental shifts in manufacturing processes, e.g., to produce heterogeneous nanowire compositions, e.g., varying in composition along their lengths.

A first example of the flexibility of the planar process methods for fabricating nanowires includes, e.g., the ability to readily vary the cross-sectional dimensions across the length of individual nanowire elements. In particular, because the nanowire element is defined from a large planar substrate, one can readily vary the width dimensions of the wire element, e.g., by patterning a wider wire in certain regions. Further, because lithographic or ablative techniques are used, and preferably, direct write techniques are used, adjusting the width simply requires varying the pattern that is either masked or written onto the substrate. As a result, one can have a single nanowire element integrated onto the insulating substrate where the nanowire element has at least two regions, where one of the regions has a different cross-sectional dimension than another region of the nanowire element. Optionally, one could provide multiple different dimensioned regions in a single wire, e.g., three, four, five, ten, twenty or more regions of varied dimensions. These different regions could vary between two, three, four, five, ten, twenty or more different width dimensions, depending upon the desired configuration of the nanowire element.

In addition to being able to vary the width of planar nanowire elements, one may alternatively or additionally wish to vary the thickness of the nanowire element. For planar process fabricated wires, this can be readily accomplished by masking off portions of the wire element and oxidizing through a fraction of the thickness of the wire element in the exposed portion(s) of the wire. The resulting oxide is then removed in an oxide etching step using, e.g., HF, to yield a thinned portion of the wire element. While etching techniques can be used and controlled to provide such thinning, these methods are not preferred, as they are less controllable than an oxidation based process. Again, by employing conventional fabrication processes and equipment in carrying out the operations described herein, such process steps would be routine for one of ordinary skill in the art. Alternatively or additionally, one could effectively thin a device through the backside depletion of the semiconductor layer, resulting in a virtual thinning of the device without use of extraneous fabrication steps.

In another example of the flexibility of planar processed devices, one can readily vary the composition of the wire elements using conventional photolithographic methods. For example, one can utilize lithographic techniques in differentially depositing and/or removing oxide and/or nitride layers from different portions of a single nanowire element. Such differential compositions provide unique capabilities for decorating these nanowires, e.g., with biomolecules for sensor applications. In particular, one might attach one type of biomolecule to one portion of a wire, e.g., having an oxide coating, while attaching another type of biomolecule (or the same biomolecule via a different mode of attachment) to the other portion of the wire, e.g., having a nitride coating. Similarly, as noted below, one could readily mask off portions of a nanowire element during ion implantation, or other doping process, e.g., vapor thermal diffusion or spin-on dopant, in order to differentially dope different portions of a nanowire element.

III. Process for Manufacturing

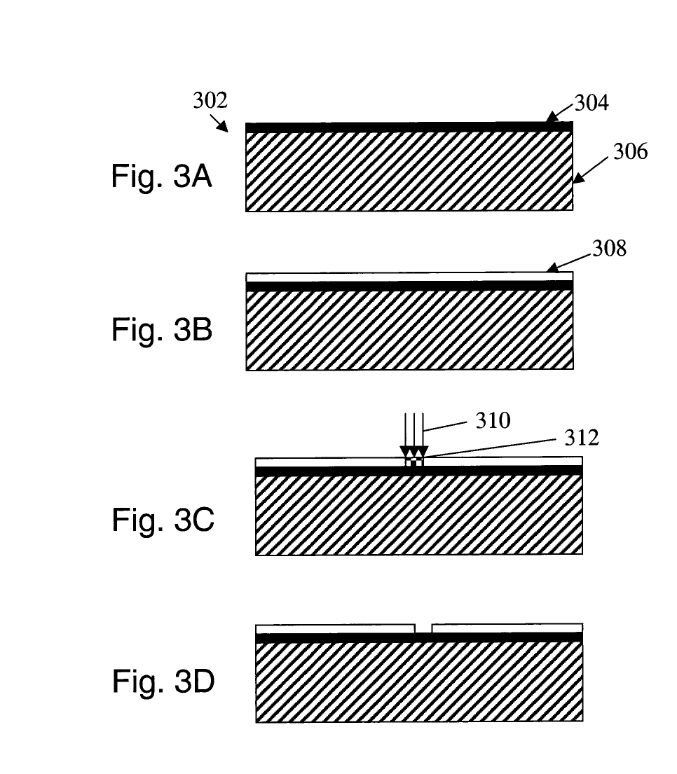

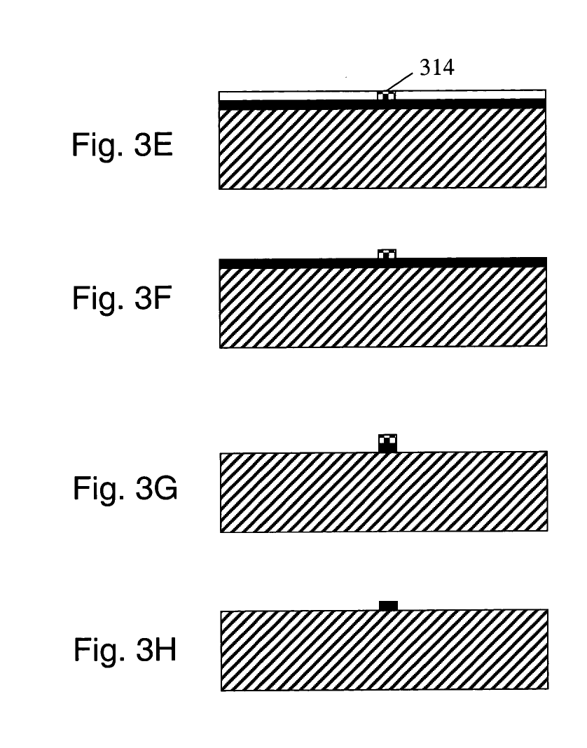

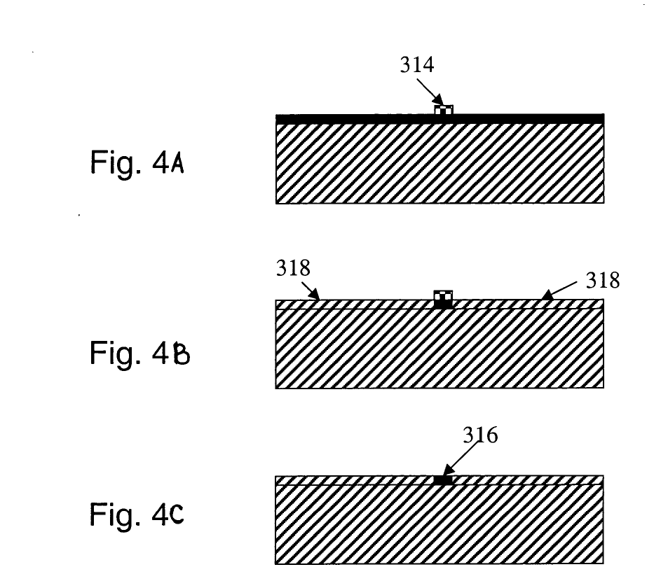

In general, the sensor elements and integrated elements of the invention are typically fabricated using conventional semiconductor fabrication processes and employing starting materials that are generally commercially available. The process is schematically illustrated in FIG. 3. Since the sensor elements described herein comprise a thin layer semiconductor element disposed on an insulating substrate, the process for fabricating such elements begins with the selection of an appropriate substrate wafer. The wafers or substrates, e.g., substrate 302, for use as a starting material in fabricating the devices of the invention include a thin semiconductor layer 304 on an insulating substrate layer 306. By a “thin” semiconductor layer is typically meant a layer of semiconductor that is less than 500 nm thick, preferably, less than 200 nm thick, more preferably less than 100 nm thick, and in many cases less than 50, 25 or even 15 nm thick. In certain preferred aspects, the semiconductor layer will be between 10 and 500 nm thick, preferably, between 10 and 200 nm thick, more preferably, between 10 and 100 nm thick, between 15 and 100 nm thick and between 15 or 50 nm thick.

A variety of different types of wafers are commercially available that meet the thickness criteria of the invention. For example, silicon on insulator (SOI) substrates are commercially available from, e.g., Virginia Semiconductor, Soitec, Inc., Silicon Valley Microelectronics, Inc., Isonics, Inc. and the like, with a semiconductor layer on a silicon dioxide insulator layer, where the semiconductor layer is thinner than 100 nm, and can be as thin as 250 angstroms (25 nm) and even down to as thin as 150 angstroms (15 nm), and even, in some cases down to 100 angstroms (10 nm). These SOI wafers are typically produced by bonding a first, high quality semiconductor wafer having an oxide layer, e.g., Si with an SiO₂ layer, to another semiconductor wafer having an oxide layer, such that the two wafers' oxide layers are mated and bonded together into a single wafer that sandwiches a single oxide layer between the high quality semiconductor layer and the other semiconductor layer. The high quality semiconductor layer is then thinned to yield the SOI wafer of the desired dimensions.

Another example of a substrate type that is useful in carrying out the invention is a SiMOX wafer where a silicon substrate is implanted with oxygen with a primary distribution at a selected depth below the wafer's surface. Annealing of the implanted substrate then produces an insulator layer (SiₓO_{y} where x/y is typically greater than 0.5) at the selected depth below a crystalline silicon layer. These substrates are generally commercially available with a variety of semiconductor layer thicknesses, e.g., from 50 to 200 nm, from, e.g., Ibis Technology Corp. (Danvers, Mass.), Sumitomo-Mitsubishi Silicon Corp., and Wacker-Siltronic Corp. (Portland, Oreg.).

Because the semiconductor layer is integral to the insulator layer in the starting substrate wafer in each of these cases, it will result in a nanowire layer that is likewise integral to the insulator layer, e.g., along the longitudinal axis of the wire. This is distinguished from semiconductor wires that are fabricated as free standing wires and later attached to an insulator layer. In the case of the nanowires of the invention made by the processes described herein, the nanowire component is molecularly uniformly attached, e.g., the full length of the nanowire component is molecularly coupled to the insulator layer.

Once a wafer is selected, it is generally coated with a lithographic resist layer 308 (FIG. 3, Panel B). In particularly preferred aspects, the resist is an e-beam resist, although for certain alternative processes, a standard photoresist can be used. E-beam resists and photoresists (both positive and negative photoresists) are widely available from commercial sources, e.g., Shipley, Inc., Hitachi, and a variety of other sources. Coating of resist on the wafer is generally accomplished using conventional methods, e.g., spin coating, etc. followed by a curing or soft baking step.

Once the resist is deposited on the wafer surface, the resist is exposed in order to define the pattern of the desired sensor element (as represented by arrows 310 in panel C of FIG. 3), as well as any connected circuitry. Exposure may be carried out using conventional photolithographic methods, e.g., where light is shone through a photomask, or by writing the specific pattern onto the resist layer, e.g., in methods employing e-beam or laser based lithography, or by a combination of methods, e.g., conventional photolithography and e-beam or high-resolution lithography. Once exposed, the resist is developed to remove either the exposed portion(s) of the resist layer, e.g., resist portion 312 (for positive resists) or the unexposed portions of the resist layer (for negative resists)(FIG. 3, Panel D). In preferred aspects, positive resists are used and the area corresponding to the sensor element and connected elements are exposed during development. Following development an etch mask layer 314, e.g., SiN, SiO, or chrome, may be applied over the exposed regions of the substrate surface to provide an etch mask layer, and the remaining resist layer is removed (FIG. 3, Panels E and F) to define the etch mask. The substrate is then etched using reactive ion etching to etch away the portions of the substrate surface that are not protected by the mask layer (FIG. 3, Panel G). The etching step results in the nanowire portion that is protected by the etch mask layer being electrically isolated from any remaining semiconductor portions of the initial substrate, except for those portions that comprise integrated circuitry to the nanowire element. The electrical isolation may involve removal of all of the semiconductor layer, or merely that portion that immediately surrounds the nanowire element. In some cases, it may be desirable to over etch a certain feature, e.g., etch beyond the edges of the masking layer, in order to define nanowire elements that are narrower than would be obtainable using the lithographic steps, e.g., e-beam lithography.

In one alternative process, as shown in FIGS. 4A-C, the wafer is processed as shown in FIG. 3, but with 4A-C in place of 3F-H. However, the semiconductor layer surrounding the defined structure 316 may not be removed, but will instead be oxidized to provide an insulator layer 318 that surrounds and insulates the defined structure from the remainder of the semiconductor layer on the substrate (not shown). In particular, instead of etching away the semiconductor layer that surrounds the defined nanowire structure, one can simply oxidize the exposed semiconductor or otherwise convert it to an insulator, e.g., silicon dioxide. For such case, the masking layer 314 will typically comprise an oxidation resistant layer, e.g., SiN, or the like. The masking layer 314 is then removed to reveal a semiconductive wire element surrounded by insulator.

In another alternative aspect, one could use standard wet chemical etching to achieve nanowire elements that are of the dimensions, e.g., thickness and width dimension described herein. In such cases, it may not be as important to start with silicon layers that are as thin as described above. In particular, one can define a strip of semiconductor on insulator that is of dimensions that are common to feature sizes in conventional wet chemical etch processes, e.g., widths of 1 μm. For example, if starting with a 1 μm thick silicon on insulator layer, one could then etch a lm wide strip. Following the definition of the initial strip, one could then isotropically etch the entire strip to reduce the strip in all dimensions, e.g., by stripping off any masking layer prior to the last etching step. By appropriately timing the etch process and/or gauging the concentration of etchant, one could precisely etch a strip that was on the scale of 10 to 100 nm deep and wide. In particular, in starting from a 1 μm wide, 1 μm deep feature, isotropic etching will etch away in both width and depth dimensions at the same time and at the same rate. By appropriately timing the etch process, one could remove the substrate from the etch bath when a desired feature size, e.g., from 10 to 50 nm wide by 10-50 nm deep is achieved. Further, by adjusting the etchant concentration, e.g., diluting the etchant, one could slow the etch process and make precise timing more reasonable, from a manufacturing standpoint. Thus, one would have a wider window during which to remove the finished product.

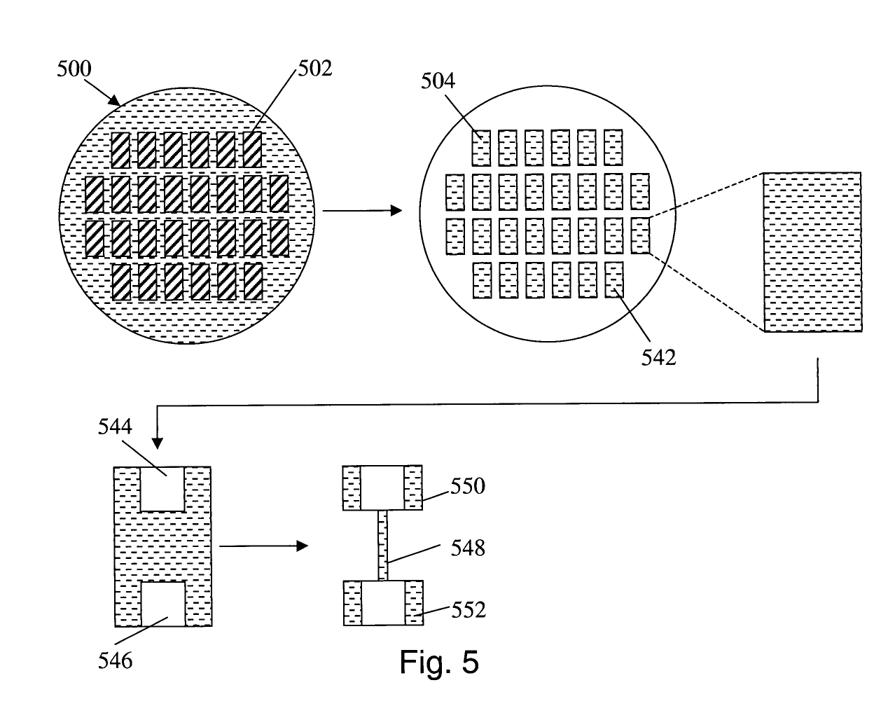

In a further alternative process, the wire element may be defined from the thin semiconductor layer using an ablative rather than lithographic technique, e.g., without requiring a resist development step. In particular, one may employ focused ion beam (FIB) processing to remove that portion of the device layer that does not correspond to the nanowire element. As with the e-beam technique described herein, this FIB method results in the direct writing of nanowire elements. In contrast with e-beam methods however, the nanowire elements are written directly into the device layer, rather than writing the pattern (or its negative) into a resist layer which is subsequently used to etch the device layer. In many cases, it may be desirable to provide a larger substrate with small device layer islands from which separate wire elements are to be fabricated using FIB methods. In particular, in starting with, e.g., an SOI substrate, one can mask off a series of small device regions, e.g., regions that are from 3 to 10 μm across, and etch away all of the unmasked device layer. This would result in a series of small semiconductor islands on the insulator layer. By reducing the working substrate to a relatively small scale by facile processes, e.g., standard wet chemical etching, one can minimize the amount of material that needs to be removed by the FIB. Additional elements could then be fabricated onto the islands, e.g., electrical contacts (front side or back side). The nanowire elements would then be defined from these small islands between the metal contacts using FIB processing. A schematic flow chart of the processing of an array of nanowire elements from a substrate is shown in FIG. 5. As shown, a standard SOI wafer 500 (e.g., as described previously herein) is provided and masked to define a series of small device layer islands 504-542 (shown in black). This is easily accomplished by exposing and developing a resist layer to mask off such islands (e.g., shown as hatched regions 502). Electrical contacts 544 and 546 are then deposited on each device layer island, and a nanowire element 548 is then carved out of the island using FIB, such that each end of the wire element 548 is connected to an ohmic contact region of semiconductor, e.g., contact regions 550 and 552. In certain preferred aspects, the metal electrical contacts and other processing steps are applied after the defining of the wire element. Following FIB definition of the wire element, in some cases it may be desirable to anneal the device layer to remedy any defect created in the wire elements by the FIB process.

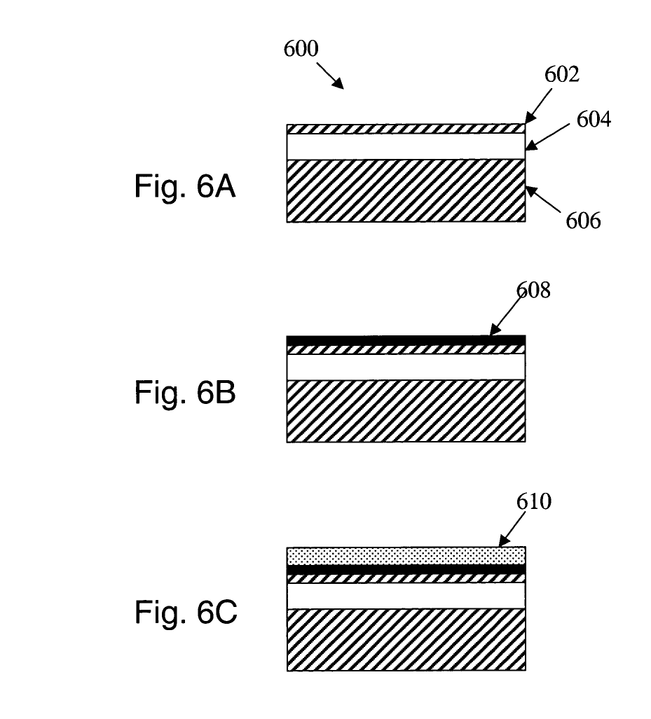

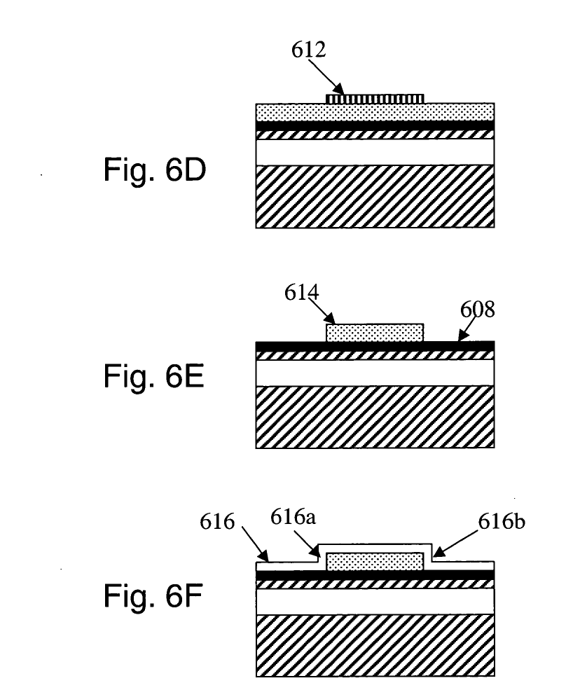

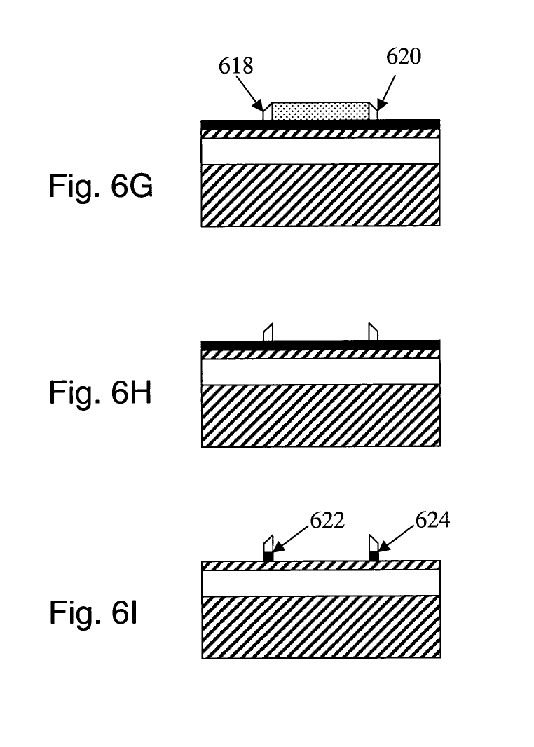

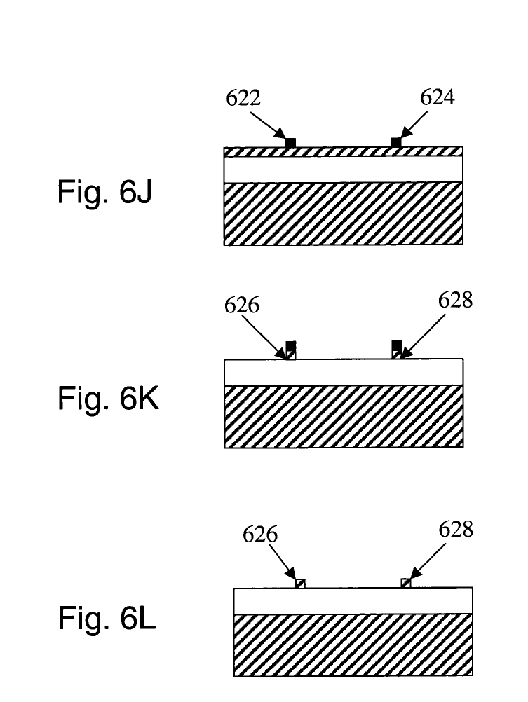

Relatedly, spacer technology may be employed to fabricate the nanowire elements of the invention from thin semiconductor on insulator, e.g., SOI, substrates, using relatively conventional lithographic techniques. Briefly, a multistep masking and lithography approach are used to first define the space between adjacent features, e.g., nanowire elements. Subsequent masking and lithography steps then define nanoscale elements at the edges or bounds of the space defined earlier in the process. An example of this process is illustrated in detail in FIG. 6 (A-L). As shown, the nanowire fabrication process starts from the same semiconductor on insulator substrate as described above. In particular, and by way of nonlimiting example, an SOI substrate 600 having a top silicon layer 602 that is, e.g., 50 nm thick or less, which forms the device or wire layer, is provided on a silicon oxide insulator layer 604 which is in turn, supported by a bulk silicon substrate layer 606. Again, as noted elsewhere herein, such SOI substrates are generally commercially available having a range of different thicknesses to the device layer. A layer of dielectric material, e.g., silicon nitride, SiO or other suitable material, 608 is then deposited over the device layer of the substrate. Although referred to hereafter as the nitride layer, it will be appreciated that any material that can serve as an etch mask during etching of a polysilicon layer, as described below, may be used, e.g., SiO, or the like. Typically, this nitride layer 608 can range in thickness, but will preferably fall in the range of 10 to 80 nm. Deposition of the nitride layer is typically carried out using, e.g., low pressure chemical vapor deposition (LPCVD) or PECVD. A masking layer 610, e.g., of polysilicon, that may be in the range of, e.g., 100 nm, is then deposited over the nitride layer 608, using, e.g., LPCVD. The nitride layer 608 is typically provided as a protective layer for the underlying device layer during subsequent etching steps, e.g., a polysilicon etching. Other masking layers may optionally be used in place of polysilicon, including, e.g., SiO, SiN, AlN, SiC and metal layers, e.g., aluminum, chrome, etc. Where other masking layers are employed in place of polysilicon, the nitride layer 608 may not be employed. A layer of a photoresist (not shown) is then deposited on the top of the masking layer 610, which resist is typically deposited by, e.g., spin coating, or other well known resist deposition methods. The photoresist may be any of a variety of different photoresists that are well known in the art of photolithography, including, e.g., positive or negative resists.

The photoresist is then exposed and developed in a pattern that will define the space between adjacent wire elements (FIG. 6, Panel D). For example, if multiple wires are to be fabricated in an array, where each wire is to be positioned, e.g., 0.35 μm apart, then the resist is developed to provide a resist layer 612 that is 0.35 μm wide. With the resist spacer layer 612 in place, the polysilicon layer is then etched using a dry etch process, e.g., plasma etching or RIE, and the resist layer is stripped away to provide a polysilicon mask layer 614 defined over the nitride layer 608, where that mask layer defines the pattern of spacers between wire elements (FIG. 6, Panel E). While wet etching could be used for this etching step, such wet etching typically will not provide a sharp enough edge profile on the polysilicon mask 614 to yield well defined elements, as would be achieved using a dry etch process. Examples of such suitable processes include plasma etching using ICP or HDP processes along with passivation. Dry etch processes, such as RIE or plasma etching produces significantly more vertical sidewalls, and thus provides for more defined mask element. This etching step is controlled to avoid excessive removal of the underlying layer, e.g., nitride layer 608. Another layer 616 is then conformally deposited over the entire substrate, including the nitride layer 608 and polysilicon masking layer 614 (FIG. 6, Panel F). While in preferred aspects, this layer 616 comprises an oxide layer, a variety of other layer types may be used, including silicon nitride, silicon carbide, aluminum nitride and/or metal layers, e.g., chrome. Typically, this layer will be deposited to a thickness that corresponds to the desired width of the ultimate nanowire elements. For example, it may range from 10 to more than 100 nm in thickness. Conformal deposition may be carried out by a number of known processes, including, e.g., Low temperature oxide (LTO), high temperature oxide (HTO) or tetraethyl orthosilicate (TEOS), PECVD, MOCVD, MBE, and the like.

One possible result of dry etching of silicon nanowires is damage during the dry etch process. There is a general concern that dry etching of nanowires can introduce surface states, e.g., dangling bonds. Therefore, in some embodiments herein oxidation of the silicon wires after the dry etch process can be used to incorporate any damaged layer which can then be chemically removed with, e.g., HF, etc. Additionally, hydrogenation can also optionally be used to passivate dangling bonds and the like. For example, etched nanowires (e.g., ones having undesirable surface states such as dangling bonds) can be exposed to a hydrogen or NH₃ plasma at high temperature or in a plasma chamber after dry etching or even after wet chemical etching of the oxide layer.

The upper facing surfaces of the conformal oxide layer are then anisotropically etched away using, e.g., reactive ion etching (RIE) to remove the thinner upper layers of the oxide layer 616 without reducing the thickness of the sidewall layers (e.g., 616a and 616b), to yield the polysilicon mask layer with SiO spacers 618 and 620, at each edge that will be approximately 50 nm wide, e.g., the same as the thickness of the oxide layer (FIG. 6, Panel G). By using an anisotropic RIE process to remove the oxide layer, the etching process can be controlled to remove only the thickness of the oxide on the upper facing surfaces, e.g., those surfaces that are directly impinged by the reactive ion beam. This leaves thicker regions, (as viewed from the direction of the ion beam) e.g., the sidewall oxide layers, intact. The thickness of the unremoved layer, e.g., spacers 618 and 620, then defines the width of the oxide mask. By varying the thickness of the oxide layer, one can readily vary the thickness of the masking element remaining after the RIE process, and ultimately, the wire element defined therefrom.

The polysilicon mask element 614 is removed to leave the SiO spacers 618 and 620 (FIG. 6, Panel H) which then form a mask layer for the underlying nitride layer 608. The polysilicon layer may generally be removed using conventional methods, including dry etch processes or wet etch processes employing, e.g., KOH, TMAH, or other commercially available silicon etchants, which etch silicon without attacking the oxide spacer layers 618 and 620. The nitride layer 608 is then etched away using a slower etch process, e.g., plasma or reactive ion etching, to provide a SiN mask layers 622 and 624 (beneath the SiO mask layers 618 and 620) that will define the wire elements in the device layer. The SiO mask layers 618 and 620 are then removed using, e.g., a conventional wet chemical etch process to reveal just the nitride mask layers 622 and 624 disposed over the device layer where the nitride etch mask layer corresponds to the width and location of the wire elements, e.g., 50 nm thick and spaced 0.35 μm apart. The substrate is then dry etched to remove the device layer around, but not beneath the nitride etch masks, to produce nanowire elements 626 and 628, disposed on insulator layer 604, where the wires are of the desired width, e.g., 50 nm, and spacing, e.g., 0.35 μm apart (FIG. 6, Panel K). Finally, the nitride layers 622 and 624 on the upper surface of the wire elements are optionally removed to provide crystalline nanowire elements disposed on an insulating substrate (FIG. 6, Panel L).

The above described fabrication process represents one example of a simple fabrication process for a device according to the present invention. It will be appreciated that the manufacturing process can include a variety of additional or alternative methods, depending upon the nature of the overall sensor and system to be produced. By way of example, where complex integrated circuits are included in the fabrication process, as will be useful in a variety of applications, conventional semiconductor processing may be used in addition to the steps used to produce the basic sensing element. This includes various conventional photolithographic, etching, oxidation, sputtering and other steps generally employed in the overall fabrication of semiconductor devices. For example one can readily use conventional processing techniques to grow additional surface layers on the exposed surface of the nanowire elements, e.g., to provide sensing surfaces of desired chemistries, such as, oxide or nitride surfaces, or to provide insulating or other layers over all, or portions of, the nanowire component of the device (where structurally separated from the sensing surface).

In addition to the basic processing of the nanowire element, one can selectively modify or differentiate different portions of the nanowire element using conventional fabrication techniques. For example, one can differentially dope different segments along the length of the nanowire component using conventional masking and doping techniques. Such methods could be used to provide p-n junctions along the length of the nanowire by masking a portion of a p-doped semiconductor nanowire and n-implanting it in the exposed region. In addition to differentially doping different portions of individual nanowire elements, or different nanowire elements in an array of elements on a single substrate, one can also selectively dope the upper portions of planar nanowires, e.g., by only implanting to a desired depth, such that the entire wire element or selected portions of a wire element comprises a p-n junction spanning the entire length of the wire or a desired segment.

As will be appreciated, the ohmic contacts in some embodiments herein present challenges due to, e.g., contact resistance (R_{c}) between the nanowires and the metal plates involved in the junction. For example, optimization is often desired of the contact resistance between the silicon compound of the nanowires herein and the metal plates. Additionally, control of silicidation of the nanowires at the junction area is also desirable.

Similar concerns of contact resistance and silicidation occur in non-nanowire contexts as well (e.g., in construction and use of semiconductors generally, etc.) and those of skill in the art will be familiar with dealing with such concerns. However, the nanowires and nanowire devices/structures of the instant invention present unique challenges due to their extremely small size and to their three-dimensional conformations.

To address contact resistance, several typical approaches have been used in non-nanowire situations. For example, the barrier height (i.e., the Schottky barrier) which exists between the two materials at the junction can be altered by, e.g., selection of different materials to form the junction. Metals such as titanium, platinum, chroming, aluminum, copper, gold, etc. can be selected in conjunction with specific dopants in the silicon layer to produce a smaller barrier height. Additionally, the method of production of the junction can also influence the barrier height through its impact on the creation of suicides, etc. which, in turn, affect the material forming the junction. Thus, the silicon compound in the junction can be suicides such as, e.g., TiSi, PtSi, CoSi, NiSi, TaSi, etc. Such suicides can comprise a layer between the metal plate area and a silicon layer beneath. Those of skill in the art will be quite familiar with such materials and their influence on barrier heights at junctions.

The contact resistance can also optionally be reduced by decreasing the depletion width associated with the junction, e.g., through selection of the specific type and amount of dopants used in the silicon aspect. Again, such techniques of altering contact resistance in ohmic junctions through changes in doping, etc. are well known to those of skill in the art. It will be apparent that the above selections of materials, dopants, etc. can be used in various embodiments herein in regard to the nanowire/metal plate ohmic junctions of the invention.

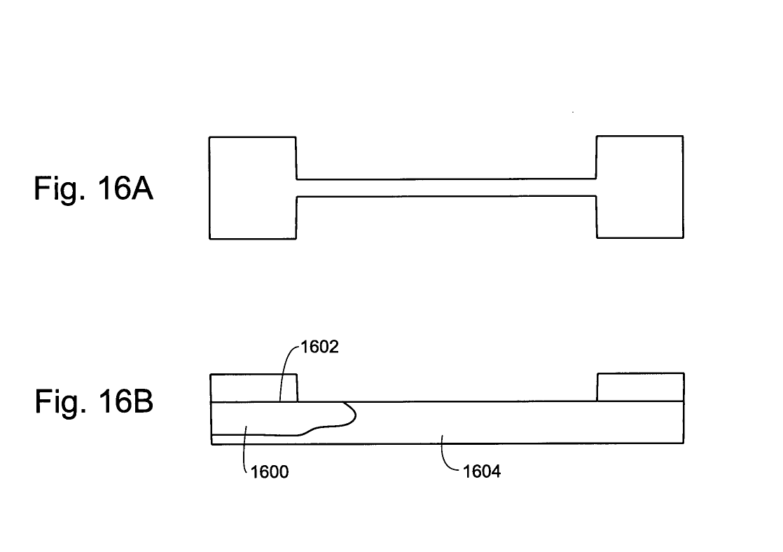

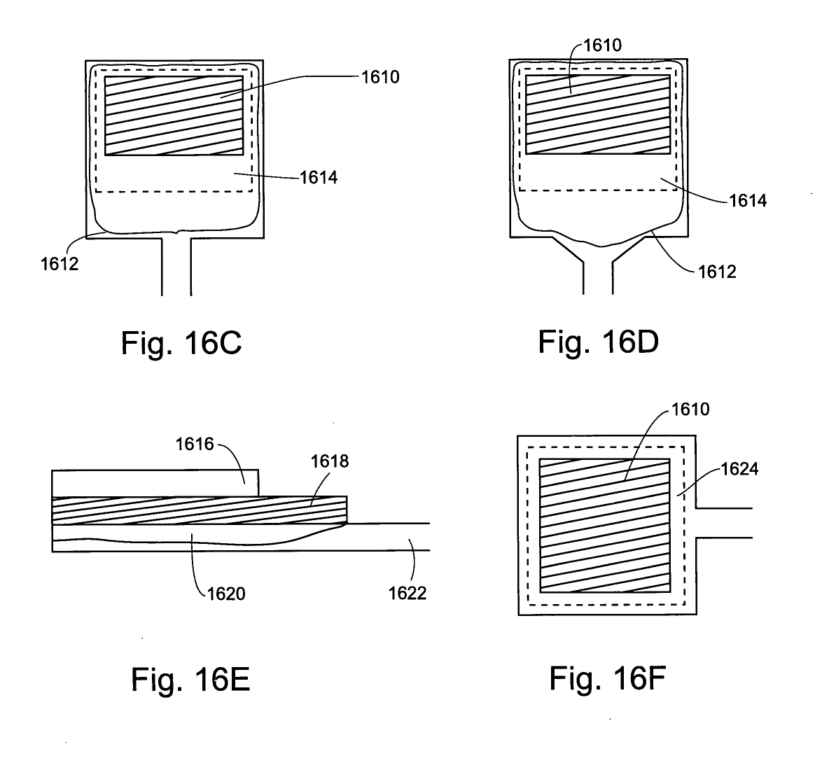

However, as mentioned previously, even though such concerns exist in other contexts, nanowires and nanowire constructions present unique situations which can be addressed through unique responses herein. For example, see, FIG. 16. As can be seen in FIG. 16B, the side view of a planar nanowire as seen in 16a, the depletion width 1600 created by the material junction 1602 can extend in some embodiments across a large percentage of the nanowire and for a considerable distance down into the nanowire 1604 itself. In some embodiments, the presence of such depletion width area can lead to problems in measurement in the extremely fine nanowire FETs herein. In more traditional semiconductor applications, the depletion width from a similar junction would affect a small percentage of the whole, and, thus, would typically be of less concern.

Therefore, some embodiments herein are configured in fashions similar to FIGS. 16C-16F. As can be seen in FIGS. 16C-16F, the metal plate aspect 1610 of the material junction can be recessed or set-back, further away from the actual nanowire. Thus, the depletion width area 1612 caused by the junction of the metal/silicon compound will not extend into the actual thin nanowire area, but instead will only extend into the broader shield or pad area of silicon compound. Additionally, as shown in FIG. 16D some constructions can have a tapered “neck” leading to the planar nanowire, thus, further recessing the metal plate from the thin planar nanowire region. In FIG. 16, a n or p doped region 1614 also exists.

Another problem faced in junction areas is silicidation of the silicon layer through contact under various conditions with the metal plate. For example, silicon nanowires annealed with titanium plates can result in creation of titanium silicide (i.e., consumption of the silicon nanowire). In nanowire construction, creation of such silicide presents problems in that, because of the small size of the nanowire, the silicon under the metal plate could be completely consumed, thus, resulting in pure silicide. Thus, the stress built in silicides, the different coefficients of thermal expansion between silicides and silicon materials, and the small contact area between the silicide and the silicon nanowire can lead to weakening and eventual breakage of the nanowire. To combat silicidation, some embodiments herein comprise a “sacrificial layer” 1618 between the metal plate 1616 and the silicon layer (nanowire) 1622. Such sacrificial layer is, thus, the area which undergoes silicidation, not the silicon of the nanowire. See, e.g., FIG. 16E. The sacrificial layer can comprise poly or amorphous doped silicon or the like. Additionally, the sacrificial layer can also, in some embodiments, reduce the silicon consumption under a metal pad/plate. It will be appreciated that various embodiments herein can comprise both recessed plates 1610 and sacrificial layers 1624 in their construction. See, e.g., FIG. 16F. The recess or set-back of the metal contact plate and the use of sacrificial layers between metal plates and silicon nanowires can also be utilized in other nanowire constructions in addition to the planar nanowires herein. For example, nanowires with a cylindrical aspect, e.g., grown from a silicon syntheses (see, e.g., Gudiksen et al, J. Am. Chem. Soc. 122, 8801-8802 (2000) and Cui et al., App. Phys. Lett. 78, 2214-2216 (2001)) which are harvested and placed horizontal to a silicon surface can also be used as FETs, etc. The above measures, especially use of sacrificial layers, are also utilized with such cylindrical nanowires. As explained in more detail below, the nanowire constructs of the invention can be created in a number of ways. Correspondingly, a number of different ways to create the recessed or set-back plates and/or sacrificial layers described above also exist. For example, a shallow junction contact comprising a recessed plate can be created as follows. First, a layer of silicon dioxide sandwiched by Si layers can have a layer of SiO deposited upon it (e.g., through thermal oxidation). Next a contact window can be created in the SiO layer through lithography, photoresist strips, etc. Next, ion implantation/gas diffusion can create a dopant region in the upper silicon layer underneath the contact window. Next, a sputter deposit of metal (e.g., a silicide metal), typically Ti can be laid over the entire surface of the wafer. Annealing (e.g., RTA, laser, or thermal) acts to form a silicide (e.g., from the metal previously deposited) in the Si layer beneath the contact window (i.e., on top of the doped Si region). The silicide typically does not form between the metal and the silicon dioxide in the other areas. See, above. Next, a metal etching (typically wet etch, but optionally dry etch) can remove non-silicide metal on the top of the silicon dioxide layer, thus, leaving the silicide layer on top of the doped silicon layer that existed under the contact window. Next, a contact metal (e.g., the metal plate) can be defined and sintered (e.g., via e-beam evaporation, etc.) as can a diffusion barrier metal layer underneath the contact metal. The contact metal layer formation can be done either before or after any nanowire is defined. If a sacrificial layer is to be included, the majority of such steps (or similar steps) can be done with the following modifications. An n or p doped polysilicon layer is overlaid onto the top of an SOI. Next, a pad area of the sacrificial material is defined through, e.g., lithography, etching, etc. on top of the upper silicon layer. Similar steps as previously described can then follow to form the metal plate, etc. In some embodiments, the dopant step and the creation of the sacrificial layer can be done together, e.g., the dopant will diffuse out of the sacrificial layer into the Si layer below. Again, while the above is an example, it will be appreciated that the various planar nanowire constructions herein are optionally created through a number of different process and steps.

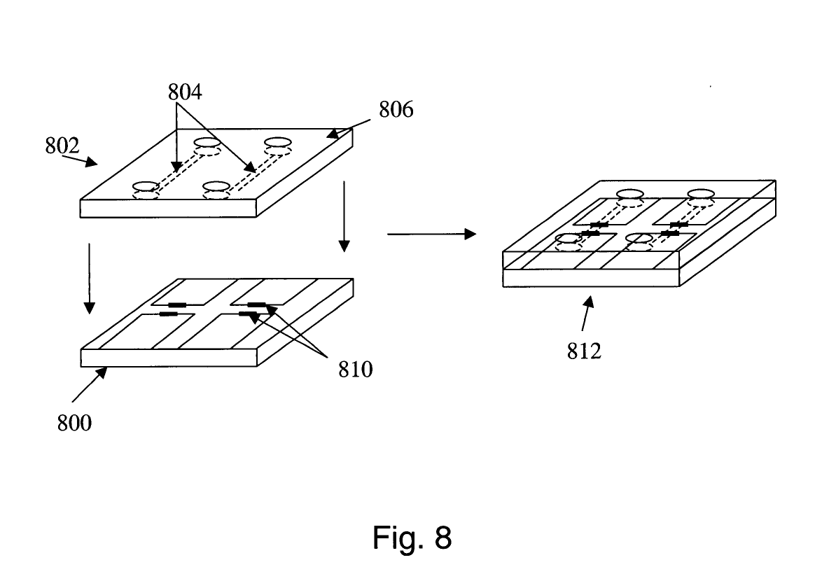

Fabrication also will typically include packaging processes, for providing the sensor element and its integrated circuitry in a commercially useful embodiment. Typically, such packaging includes providing the nanosensor with appropriate interface components for coupling the sensor to ancillary instrumentation, e.g., power supplies, computers, etc. Such packaging also may include providing the sensor integrated into fluid vessel, e.g., a well in a multiwell plate, or in a fluidic conduit or channel in, e.g., a microfluidic device, that may be provided upon the same substrate or on an additional substrate that is overlaid and bonded to that which comprises the sensor element. Again, because the present invention employs conventional semiconductor processing techniques to produce the sensor devices described herein, conventional packaging processes also may generally be used. A simplified schematic example of the integration of a sensor array of the invention in a microfluidic device is provided in FIG. 8.

IV. Structural Attributes of Nanowire Based Sensor Elements, Devices and Systems

A. Sensor Elements and Devices Incorporating Them

As noted above, the basic structure of the sensing elements of the invention comprises a thin nanoscale wire, or nanowire, of semiconductor material disposed upon and integral to an insulating substrate, where the nanowire is electrically coupled to a sensing surface that has a functional moiety coupled thereto or otherwise associated therewith. This is schematically illustrated in FIG. 2. As described in greater detail below, the “nanowire sensing element” denotes that portion of an overall device that includes an active sensing surface that is capable of being exposed to and interacting with, or binding to, an analyte of interest, and a nanowire element that is electrically coupled to that surface. As used herein, the “sensing surface,” also referred to herein as an “active surface,” denotes a surface of a solid support that comprises a functional moiety or moieties that are capable of interacting with one or more analytes of interest.

By “integral to an insulating substrate” is meant that the semiconductor nanowire is directly coupled to the insulator layer without any intermediate chemical linking groups, e.g., as a silicon layer and its oxidized surface are integral. This is contrasted with, and specifically excludes, semiconductor nanowires that are chemically attached to insulator surfaces via an intermediate chemical linker that is attached at one point to the insulator and at another point to the semiconductor nanowire, e.g., via amine groups on aminated surfaces of an insulator layer.

By “electrically coupled” is meant that the nanowire component is coupled to, or otherwise positioned such that, a chemical compound or analyte having a localized charge density that interacts with the functional moiety that comprises the sensing or active surface, is capable of causing a detectable shift in the electrical properties of the nanowire component. Such electric coupling may comprise direct contact between the semiconductive nanowire and the sensing surface, or positioning such that the sensing surface and nanowire component are not in direct contact, but are in sufficient proximity that an electrical characteristic of the analyte exerts an electrical effect on a property of the nanowire component. As will thus be appreciated, the description of the sensing element as including a sensing surface electrically coupled to a nanowire includes a variety of different structures, including: simple nanowire elements whose surfaces have been coated or treated to provide the active sensing surface, e.g., coated, derivatized, etc.; nanowires that are coated with intermediate layers, e.g., insulators, metals, polymers, etc. which are in turn coated or treated to provide active sensing surfaces; and nanowires that are coupled to a non-nanowire sensing surfaces, provided that the sensing surfaces are sufficiently proximal to the nanowire element so as to induce an analyte induced effect on the conductance of the nanowire.

The nanowire element included in the sensors described herein is typically less than 500 nm thick (e.g. has a depth dimension orthogonal to the insulating substrate), preferably, less than 200 nm thick, more preferably less than 100 nm thick, and in many cases less than 50, 25 or even 15 nm thick. In certain preferred aspects, the nanowire element will be between 10 and 500 nm thick, preferably, between 10 and 200 nm thick, more preferably, between 10 and 100 nm thick, between 15 and 100 nm thick or between 15 and 50 nm thick. The length and width dimensions of the nanowire element, e.g., the dimensions running parallel to the insulating substrate, can vary depending upon the application, and the relation of the nanowire to the sensing surface. In certain aspects, where the sensing surface is disposed over, or integral with, the exposed surface of the nanowire, the width of the nanowire, and thus the sensing surface, can generally vary depending upon the desired application. For example, in certain preferred aspects, e.g., where the ability to sense low numbers of binding events is desired, the sensing element is typically less than about 500 nm in width, preferably, less than about 100 nm in width and more preferably less than about 50 nm in width and in some cases down to 15-30 nm in width, so that each discrete binding event will produce a larger effect on the nanowire component. For other applications, e.g., where the number of binding events is not expected to be limiting, e.g., higher concentrations of analyte, much larger widths could be readily used, e.g., up to 10 μm or wider. In fact, the ability to vary the width of the nanoscale sensing element provides a significant advantage over typical free standing nanowire sensors, as increasing the sensing surface area will generally serve to increase the signal to noise ratio of the sensor.

The length of the sensing element also will depend generally upon the application to which the overall device is to be put. For example, the length may be defined by the working or sensing space of the device, e.g., the dimensions of a fluid reservoir into which samples are deposited or otherwise move. Alternatively, the sensing element length may be more application specific. For example, for applications where analyte concentration is expected to be low, e.g., requiring more active surface area for a sensor, longer sensor elements are preferred, while for applications in which analyte concentration is higher, shorter lengths can be used. Briefly, by increasing the active surface area of the sensor, e.g., the surface upon which binding agents are coupled, one can increase the likelihood of binding of less concentrated analyte molecules. For more concentrated solutions of analyte molecules, a smaller active surface area will generally suffice to ensure the occurrence of sufficient binding events. In general, the length of the sensing element is from about 0.1 μm to as long as 10 mm or longer. As will be appreciated, both length and width of the sensing element can be varied depending upon whether one is desirous of detecting single or few binding events, or detecting large numbers of binding events from low concentration analytes or high numbers of binding events for higher concentration analytes.

As noted above, the active or sensing surface of the sensor element includes a functional moiety coupled to that surface that provides for the ability to identify an analyte of interest. As used herein, the term “functional moiety” denotes a chemical or structural element, and preferably, a chemical element, that is disposed upon and part of the sensing surface, that is capable of interacting with the analyte of interest in any manner that allows a localized charge density of the analyte of interest to induce a shift in the conductance of the nanowire portion of the sensor. While typically the functional moiety comprises a chemical or biochemical compound that specifically binds or otherwise associates with the analyte of interest, operability of the devices of the invention does not necessarily require the occurrence of a binding event, whether covalent or through other noncovalent binding. The functional moiety can be specific to a particular molecule or molecular component as the analyte of interest, or it can be more generic, e.g., to interact with and identify broader classes of molecules, e.g., amines, metal ions, phosphates, carbohydrates, etc.

Examples of functional moieties for use in accordance with the present invention include chemical moieties that are capable of interacting with analytes of interest, such as metals, metal dihydrides, metal oxides, nitrides (e.g., silicon nitride), and biochemical compounds. Particularly useful biochemical compounds include, e.g., nucleic acid probes (DNA, RNA or nucleic acid analogs, e.g., PNA) that are complementary to nucleic acid sequences of interest, receptor molecules that interact with ligands of interest, ligands that interact with receptors of interest, antibodies or antibody fragments, antigenic epitopes, chelators, metal ions, e.g., di- and trivalent metal ions for interacting with phosphorylated compounds etc., binding proteins, and the like. In particularly preferred aspects, the functional moieties will be uncharged, so as to eliminate any effect of such charge on the detection event, e.g., to reduce noise levels.

In certain preferred aspects, the sensing surface is provided upon the exposed surface of the nanowire component, e.g., that surface that is not connected to the insulator layer, by providing the functional moiety coupled to or otherwise associated with that exposed surface. Together, the functional moiety and exposed surface of the nanowire comprise the sensing surface of the sensing element. In such cases, the functional moiety may be directly linked to the nanowire, e.g., through silane groups, or it may be coupled via a linker molecule.

In alternate preferred aspects, the sensing surface is provided structurally decoupled from the nanowire element, provided that the sensing surface remains electrically coupled to the nanowire element. For example, in some cases, an intermediate structural layer may be provided over the exposed surface of the semiconductor nanowire element, and the functional moiety is then either a part of, or is structurally coupled to, the exposed surface of that intermediate layer, rather than directly to the semiconductor nanowire element. Examples of such layers include, e.g., oxide layers, nitride layers, metal layers, metal oxide layers, metal dihydride layers, self assembled monolayers, polymer layers, etc.

Exemplary sensor configurations include semiconductor nanowire elements that have metal layers sputtered or vapor deposited thereon to form the sensing surface and provide the functional moiety. For example, platinum or gold layers are optionally provided over a semiconductor nanowire element to provide for the measurement of redox potential of solutions. Similarly, metal dihydride layers would be provided for use in detecting and measuring hydrogen gas concentrations (see, U.S. Pat. No. 6,265,222). Metal oxide layers, e.g., SnO₂, are provided for use in detecting other gases, e.g., carbon monoxide and carbon dioxide. Similarly, silicon nitride layers provide particularly useful sensing surfaces for pH detection and measurement (see, e.g., U.S. Pat. No. 5,496,607 to Parce et al.). In particularly preferred aspects, such nitride layers are provided over an intermediate thermal oxide layer that is disposed over the semiconductor element. Such pH sensing surfaces are also optionally used in conjunction with a hydrophobic membrane disposed over the sensing surface that retains a particular ionophore (e.g., specific for Na⁺, Ca⁺⁺, Li⁺⁺, K⁺) in the space between the membrane and the wire. Such sensors then function to detect relative concentrations of the ions of interest. A variety of other chemical sensor configurations would be readily apparent to one of ordinary skill in the art based upon the instant disclosure.

In at least one particularly preferred aspect, a semiconductive nanowire element is provided with a metal coating or layer that is disposed between the wire element and the analyte containing fluid. In particular, in some cases, a contiguous floating metal layer, e.g., a metal layer disposed over an oxide layer on a wire element, is provided as a portion of the sensing surface. In particular, where a nanowire element employs a simple oxide layer, e.g., a native or grown oxide layer, it can result in sensor signal drift, either due to erosion and penetration of the oxide by sodium ions, e.g., that are present in most bioassay solutions. In particular, the high density of silanols present on the surface renders the sensor pH sensitive from proton binding or dissociation which decreases the sensor's sensitivity to other charged species of interest. This latter effect is generally dealt with by capping as many of the titratable silanols as possible through known capping chemistries. While this solution may prove somewhat effective, it is generally short lived, and results in only a fractional decrease in pH sensitivity.

In accordance with the preferred aspects of the invention, however, a metal layer is deposited over the gate region of the sensor element, e.g., over the thin portion of the nanowire element, as an island, e.g., not directly electrically coupled to the source or drain. The metal layer provides a suitable barrier for the silanols on the oxide layer surface, while simultaneously providing a conductive path between the solution and the oxide layer over the gate. Further, because the metal layer is conductive, it may be provided at any of a variety of thicknesses, in order to ensure adequate coverage of the oxide surface. A variety of different metal sensing surfaces may be employed in accordance with this aspect of the invention, including, e.g., gold, platinum, palladium, iridium, indium, tantalum, etc. Further, as noted previously, these metal sensing surfaces may be provided directly on the gate portion of the nanowire element, or they may be separate from the gate region, but electrically coupled thereto, e.g., where the metal layer is disposed partially over the nanowire element or gate and partially separate from the gate, but where the sensing surface, per se, is disposed on the portion separate from the gate. Typically, the metal layer will be deposited upon an adhesion layer, e.g., another metal layer deposited on the semiconductor substrate or gate. In particularly preferred aspects, the metal layer comprises a gold layer. In particular, the use of a gold metal layer provides readily employable coupling chemistries for coupling functional groups, e.g., binding agents to the metal layer to form the sensing surface. Specifically, thiol chemistries are readily employed for binding functional groups onto gold surfaces and would be readily employed here for, e.g., coupling nucleic acids, proteins, etc. to the sensing surface.

In some cases, it may be desirable to provide a protective layer over the gold or other metal sensing surface, in order to prevent adverse effects from redox species present in the solution that is to be analyzed, that can couple to the gold layer. In such cases, the coupling chemistry employed to couple functional groups can employ long alkyl chains, e.g., C10 or greater, to form a hydrophobic self assembled monolayer (SAM) to prevent such redox species from contacting the gold surface.

This metal layer that forms a part of the sensing surface is preferably disposed upon a metal adhesion layer. For example, a gold sensing surface layer is typically disposed upon an adhesion layer of e.g., chromium, titanium or alloys thereof.

As mentioned previously, in sensors such as in the present invention, solutions are often used to process the molecules or chemicals of interest. These solutions typically contain ions which can interfere with electrical measurements as well as cause unwanted electrochemical reactions involving the metal conduction traces.

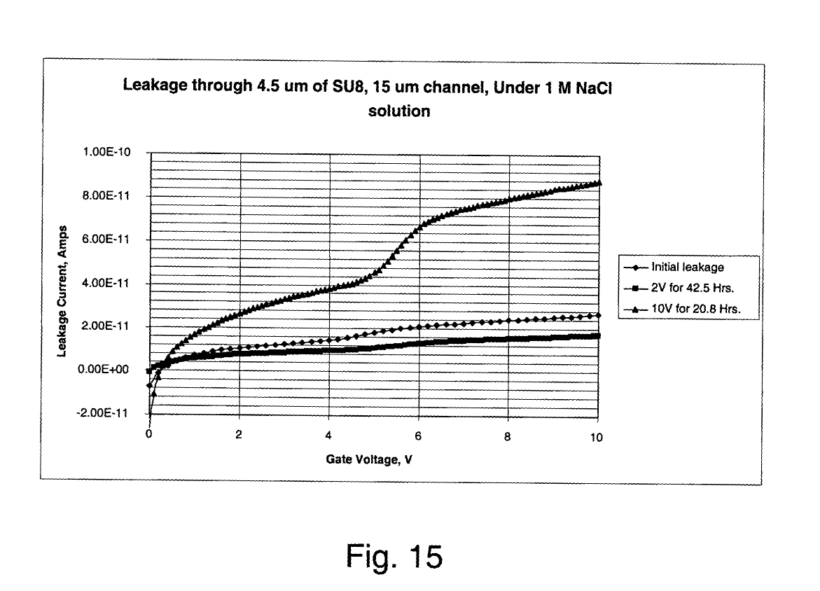

Thus, a dielectric (e.g., a polymeric film, etc.) encapsulating, e.g., the non-active areas of the sensors, prevents moisture and ions from reacting with the metal traces and other dielectric films. If salt or other similar solutions come into contact with metal trace lines while the traces are exposed to electrical voltages, unwanted electrochemistry can take place and, in worst case scenarios, the metal traces can oxidize and dissolve away. Due to their properties in protecting the areas (e.g., the electrical contacts) from problematic solutions dielectric materials for encapsulation can include, e.g., SiN, SiO, SiC, AlN, AlO, etc. or organics such as polymers with a high degree of cross-linking or those comprising a monomer with a minimal number of polar groups. An example of such material is SU-8, an epoxy monomer. Of course, SU-8 is an illustrative but not limiting example. Other types of films and compositions are also optionally used (e.g., polyimide, BCB (benzocyclobutene), etc.), depending upon, e.g., specific reaction parameters such as composition of the sensitive areas, the solutions used, etc. Furthermore, the coating, e.g., polymer, is optionally treated after it has been applied to a surface, thus, changing its properties. Additionally, if fluorescence detection is to be used in any area of the sensor (even in a subsidiary fashion, e.g., to track solute concentration, etc.), then the encapsulation material should also exhibit a very low of, or no, background fluorescence. See, Example 2 and FIG. 15, below.

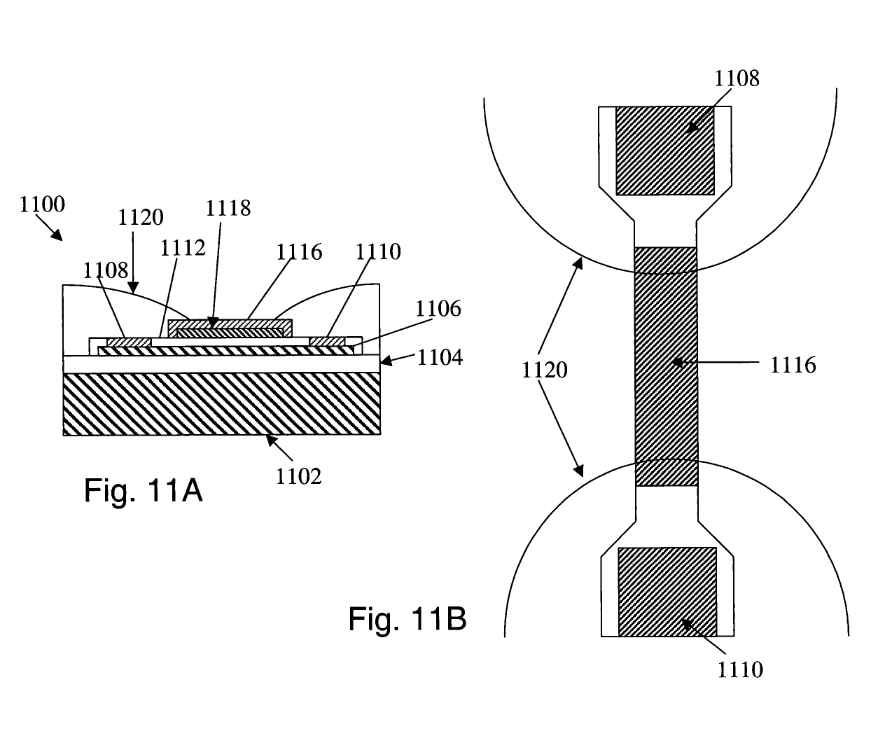

An example of a device according to this aspect of the invention is shown from a top and side view in FIG. 11A. As shown, the basic device 1100 comprises the various components of the devices described elsewhere herein, including a substrate layer 1102 that includes an insulator upper surface 1104. A nanowire element 1106 is integrally disposed upon the upper surface of the insulator layer 1104. Electrical contacts 1108 and 1110 are disposed in electrical contact with different ends of the wire element 1106. An oxide layer 1112 is provided over the wire element. In accordance with the instant preferred aspects of the invention, a metal sensing surface 1116 is provided as an island on the upper surface of the nanowire element 1106, e.g., electrically insulated from electrical contacts 1108 and 1110. As noted previously, a metal adhesion layer, e.g., layer 1118 is provided between the metal sensing surface layer 1116 and the oxide layer 1112 over the nanowire element 1106. In order to shield the electrical contacts 1108 and 1110 from fluid that contacts the sensing surface 1116, a water impermeable polymer layer 1120, e.g., SU-8, or the like, is deposited over the electrical contacts and extending to cover the exposed oxide layer 1112.

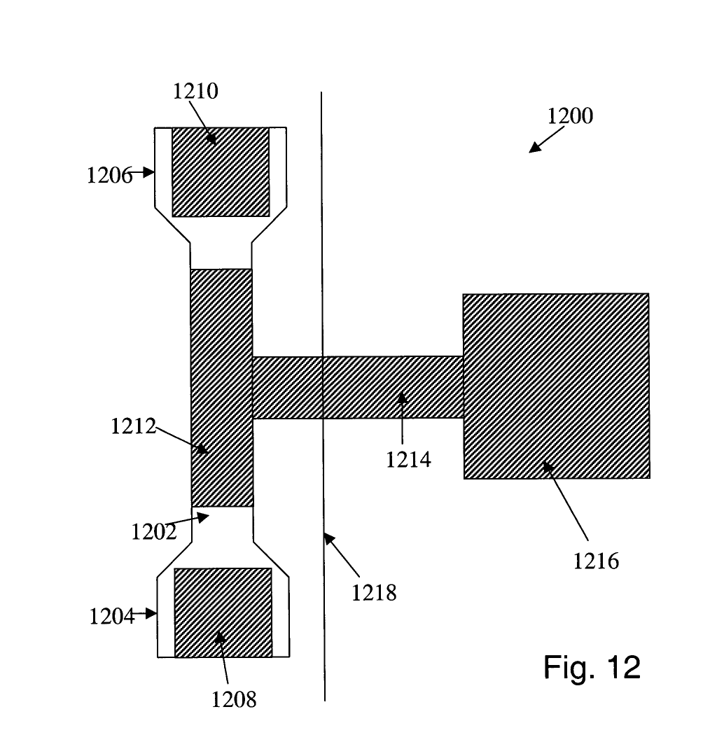

An example of a device according to this aspect of the invention, where the sensing surface is separate from, but electrically coupled to the nanowire element, or gate, is shown in FIG. 12. In particular, the overall device 1200 includes a nanowire element 1202 that has at each end an enlarged contact region 1204 and 1206, upon which are deposited metal contact pads 1208 and 1210. A metal island 1212, e.g., as shown in FIG. 11, is provided over the gate portion of the nanowire element. A metal trace 1214 then electrically couples the metal island 1212 to the metal layer sensing surface 1216, upon which are provided functional groups, e.g., binding moieties, etc. A water impermeable barrier 1218, e.g., Su-8, is disposed over the wire element and metal contacts to prevent contact with the fluid of interest during operation. Again, because the metal sensing surface 1216, trace 1214 and metal island 1212 are conductive, the distance of the sensing surface from the gate region of the nanowire element has little effect on the sensitivity of the gate to electrical effects at the sensing surface. Further, fabrication of devices, either in accordance with FIG. 11 or FIG. 12, relies upon manufacturing processes that are routinely employed in the microelectronics industry. By way of example, following fabrication of the wire element, a metal pattern may be provided as shown by, e.g., sputtering and lift-off techniques, etching methods, etc. for both the adhesion layers and the overlaying metal layer, e.g., gold, platinum or the like. It will be appreciated that the inventive nature of this aspect of the invention is not limited to gate regions that are of nanowire scales, but also includes use of more conventional size, e.g., micro-scale, transistor elements, where the sensing surface is electrically coupled to the gate region of the conventional sized transistor element.

For biochemical analyses, the structural configuration of a sensor includes a semiconductor nanowire element having biochemical functional moieties coupled to it. The functional moieties may include any of a variety of molecules that specifically interact or bind with other biochemical molecules of interest. In its simplest form, such agents include one member of a complementary binding pair of molecules, such as one member of a receptor:ligand pair, a binding protein:ligand pair, an antibody:epitope pair, an antibody fragment:epitope, a pair of complementary oligonucleotides, or a phosphorylated protein:multivalent metal ion pair, and the like. However, such agents also will include virtually any chemical moiety that will provide for a specific interaction with a molecule of interest, where that interaction yields a field induced change in the electrical properties of the sensor element. Sensors for detecting a wide variety of different chemical species, and their complementary binding agents for use in chemically sensitive field effect transistors (ChemFETs) are generally known in the art. In use, such compounds are typically coupled to the exposed surface of the nanowire or an intermediate layer.

One specific example of a type of biochemical sensing element in accordance with the present invention includes a planar nanowire that has nucleic acid probes coupled to its exposed surface, e.g., directly or through linker groups or an intermediate layer, e.g., an insulator layer, to provide the sensing surface. The nucleic acid probes are provided with a sequence of nucleotides that is complementary to and capable of hybridizing with a nucleic acid sequence of interest. In preferred aspects, uncharged nucleic acid analogs may be used as the functional moieties, so that the inherent charge of a natural nucleic acid probe does not mask any signal resulting from the binding event, yielding higher sensitivity for a given analysis. One example of a preferred class of uncharged nucleic acid analogs includes peptide nucleic acids where an uncharged peptide backbone is substituted for the highly charged glycophosphate backbone of natural nucleic acid, while maintaining the nucleobase side groups, and thus preserving complementarity to natural nucleic acids (see, e.g., Nilsen et al., Science 254:497 (1991); and Egholm et al., Nature 365:566 (1993), each of which is hereby incorporated by reference in its entirety for all purposes). The sequence of interest may be a sequence that is indicative of the presence of a particular gene, a particular organism, or of a particular allele for a genomic variant position, e.g., a single nucleotide polymorphism (SNP), short tandem repeat (STR), point deletion or insertion, or the like. The sensing surface is then provided disposed in a fluid reservoir, e.g., a well or fluid conduit, path or channel, such that a fluid to be analyzed will readily contact the sensing surface. Where the sequence of interest is present in the fluid sample, coupling of the sequence of interest to the probes on the sensing surface yields a detectable change in the conductance of the nanowire portion of the sensor.

Coupling of binding agents to the sensing surface, where necessary, is generally carried out through any of a variety of different, well known chemistries. For example, biomolecules are generally readily coupled to silicon surfaces through the use of siloxane derivatives to provide active functional groups to which biomolecules and other functional moieties may be readily coupled, e.g., hydroxyl or amine groups. Coupling chemistries for coupling a variety of different types of biomolecules to semiconductor surfaces have been previously described, see, e.g., U.S. Pat. No. 5,489,678, which is incorporated herein by reference in its entirety for all purposes.

In certain preferred instances, multiple nanowire-based sensors in accordance with the present invention are integrated into a single device or “array.” The multiple sensor elements may comprise the same or different functional moieties in the same or different orientations, surface concentrations, etc. For example, multiple different analyses could be performed on single samples by providing arrays of sensor elements that are each specific for different analytes of interest. For example, multiple different genetic loci could be analyzed by employing multiple separate sensor elements that are each specific for those different loci. In such cases, multiple different functional moieties are provided that are specific for the same analyte, e.g., a single nucleic acid sequence. Similarly, multiple different medical diagnostic analytes could be evaluated in a single patient sample by incorporating appropriate different functional moieties on multiple different sensing elements in a single device. For example, one could perform a panel of immunodiagnostic evaluations on a single patient sample, simultaneously by incorporating multiple sensor elements each functionalized with a different antibody or antibody fragment, e.g., specific for a different antigen or antigenic epitope. Further, multiple different sensors, while disposed in the same device, might be disposed within different fluid reservoirs, e.g., to contact different fluids. An example of such an application includes a sensor array that is interfaced with multiplexed fluid channels in a microfluidic device, such that a different sensor element is contacting fluid in a different channel of the fluidic device. This type of application would be particularly useful in analyzing multiple different samples for a single analyte of interest.

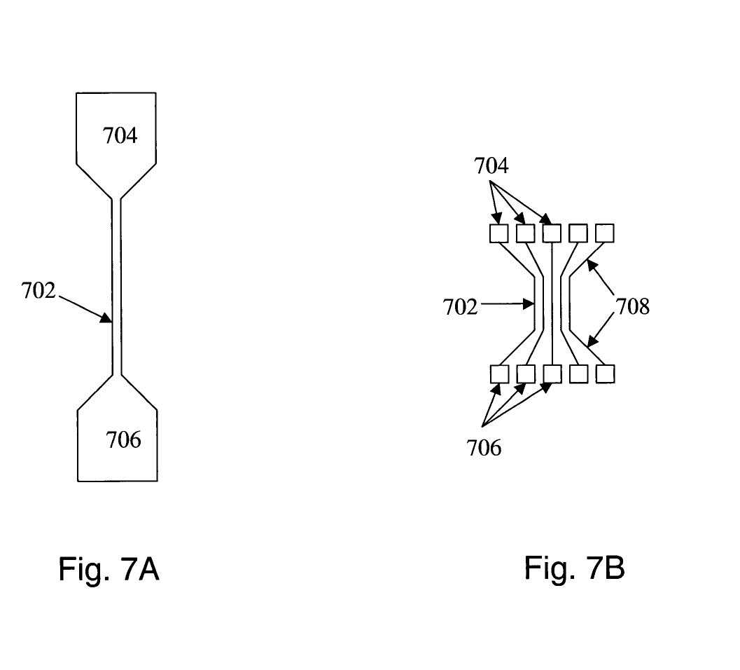

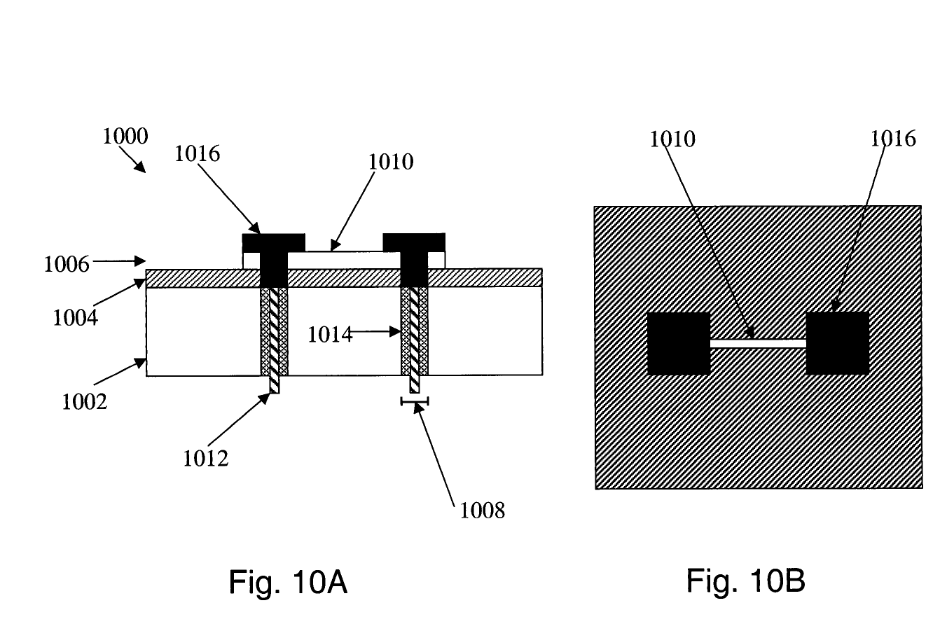

In addition to the basic nanowire component and sensing surface, as described above, preferred methods of fabrication will also provide for at least some elements useful in integration of the sensor with other components of an overall sensing system. For example, at its simplest, integration elements will include the preparation of contact points on the sensor element required to connect the sensor to its supporting instrumentation, e.g., electrical power supplies and measurement devices. One example of this type of simple integration is shown in FIG. 7. As noted previously, interfacing nanoscale based structures can provide substantial difficulties, given their small size. One example of this problem is in making electrical or ohmic contact with the sensor element. One method of addressing this issue is illustrated in FIG. 7A. As shown, the sensor element 702 is illustrated as a simple nanowire sensor. At each end of the nanowire 702 are provided enlarged semiconductor regions (on the insulating substrate). These enlarged regions 704 and 706 provide for greater ease of connecting wires or other contacts to the basic sensor element 702. Similarly methods are employed to allow for denser packing of nanowire elements in an overall device. As shown in FIG. 7B, a number of nanowire sensors 702, each having enlarged end regions 704 and 706, are provided in relatively close proximity. Connection to these closely spaced sensor elements is facilitated by providing connecting elements 708 that are directed away from each other, or “fan out,” in order to provide sufficient space for integrating larger structures or interfacing with external systems or system components. Alternatively or additionally, the electrical contacts can be provided on either the back or front side of the substrate (see, e.g., FIG. 10, and accompanying discussions herein).

In addition to the simple circuit elements described above, the processes, devices and systems described herein are particularly well suited for incorporating more complex circuitry that is integrated with the sensor element. In particularly preferred examples, the sensor devices of the invention will include protection or buffering circuits, e.g., diodes, resistors, etc., to protect the nanowire element from any transient electrical impulses occurring during manufacture or use. For array based applications of the sensors described herein, e.g., devices or systems incorporating multiple sensor elements, the devices will optionally include multiplexing circuitry, to enable facile addressing of the various sensor elements. In another example, the sensors described herein may include amplification circuitry, in order to provide easily monitored signals from operation. A wide variety of other circuit types could be readily employed in the sensors described herein, include those directly involved, indirectly involved, or completely uninvolved in the operation of the sensor elements. Such circuits are well known in the art, and by virtue of the fabrication techniques employed in practicing the invention, are readily applied to devices of the invention.

While generally described in terms of a single sensor unit, e.g., a nanowire sensor element disposed between two or more ohmic contacts, it will be appreciated that arrays of such sensors may be prepared where multiple sensor elements are provided in a single device. Such arrays may be interfaced with a single sample fluid source, e.g., a sample well or fluidic channel or they may be separately interfaced with multiple integrated sample sources. Such sensor elements may, as such, have multiple different sensing surfaces to detect multiple different analytes, e.g., as may be the case where all are interfaced with a single analyte source, or they may all have the same sensing surface, e.g., where they are interfaced with multiple different sources, but are intended to detect and/or quantify the same type of analyte in such different samples. One example of a simple array is illustrated in FIG. 8. As shown, a two device array 800 is shown interfaced with a microfluidic channel network 802. The channel network is defined as a series of grooves 804 fabricated into a planar substrate 806, which is mated to the array 800 such that a different channel traverses each of the sensor elements 810 in the array 800 to form an overall integrated fluidics array 812. In preferred systems a single array may include two, five, ten, twenty, fifty, one hundred, one thousand or more separate sensor elements. Each sensor element may be coupled to a fluid vessel, e.g., a channel or a well, with or without any other or all other sensor elements, depending upon the desired application. Further, such sensor elements can be defined in relatively high density with one thousand sensor elements being defined at a density of less than 10 cm², less than 2 cm², and even less than 1 cm². Preferably, an array in accordance with the invention will comprise sensor elements defined at a density of at least 100 sensor elements in 2 cm² or less, 1 cm² or less, etc.

Although described primarily in terms of planar integrated nanowire based sensors, it will be appreciated that a number of aspects of the invention relate to nanowire sensors, in general, and do not necessarily require that such sensors be integrated with an insulator layer, e.g., they could be used in conjunction with free-standing nanowires. Examples of such aspects include, without limitation, the use of integrated protection circuitry with a nanowire sensor, the use of nitride coatings with or without additional functional moieties attached thereto, circuitry to facilitate connection to micro or macroscale systems, e.g., fan out connectors, and a variety of other elements described above.

Although described primarily in terms of sensor applications, it will be appreciated that the fabrication methods and nanowire elements described herein can be used in a variety of applications that have been previously described for free-standing nanowires, such as in electrical circuits, LEDs, lasers, photovoltaics, etc. By way of example, a simple junction, e.g., of similar or dissimilar materials can be fabricated by fabricating nanowire elements on two separate insulator substrates, in accordance with the processes described herein. The two nanowire elements are then contacted with each other by mating the two substrates together such that one nanowire element contacts the other. Because these nanowire elements are so thin, e.g., on the order of 10 to 50 nm, the opposing substrates may be readily bonded together via thermal or anodic bonding methods, without the nanowire structure providing any interference with the bonding. Alternatively, for those substrates where the nanowire element is defined by a surrounding insulator barrier (e.g., not etched), such bonding is particularly simple. The different wires could comprise different materials, e.g., different semiconductors or differently doped semiconductor materials. For example, a simple p-n junction could be produced by fabricating a doped semiconductor nanowire on a first substrate and an n doped nanowire on a separate substrate. Bonding the two substrates together would form a p-n junction at the points the wires cross or contact.

As should be readily appreciated, all of these alternative methods of fabrication are readily practiced using the methods of the invention, and would not be easily employed in previously described nanowire based sensors.

B. Systems Incorporating Nanowire Based Sensor Elements and Devices

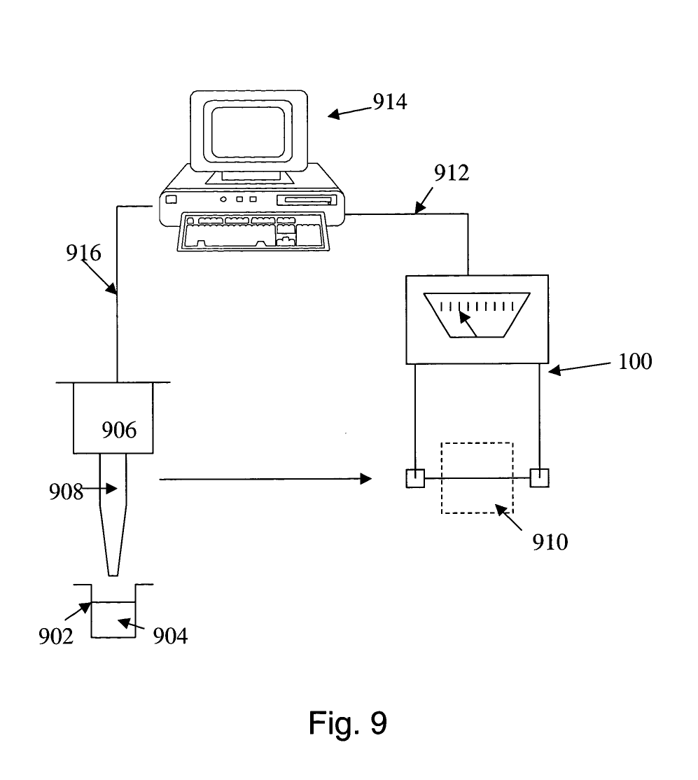

In operation, the sensor elements described above are generally integrated into an overall sensor system. An example of an overall system is schematically illustrated in FIG. 9. In addition to the nanowire element and electrically coupled sensing surface, such systems include basic electrical power supply and monitoring systems for applying and measuring electrical current across the nanowire based sensor element, as schematically illustrated in FIG. 1A. The sensing system, including the monitoring instrumentation is illustrated as system 100 in FIG. 9. Examples of basic monitoring systems, e.g., those coupled to the sensor element for measuring conductance or other properties of the wire, include a simple ohm meter. In preferred aspects, AC ohm meters are utilized as they improve the signal to noise ratio in the measurement of conductance.

In addition to the basic elements of the sensing system, the overall systems of the invention also optionally include ancillary components to automate sample accession and delivery, and provide for data storage and analysis. For example, in at least certain embodiments, fluid handling systems, e.g., pipetting system 906, are included in the overall system architecture. Such fluid handling systems typically deliver fluid sample materials to the fluid reservoirs, e.g., reservoir 910, to contact the sensing surfaces from storage vessels, e.g., vessel 902. In its simplest form, the fluid handling system is comprised of a pipettor 906 for pulling sample fluids from storage vessels 902, and expelling them into a sample reservoir 910 that is coupled to the sensing surface of the sensor element, for analysis. Such pipettor systems, including the robotic armature for moving the pipettors from vessel to vessel are generally commercially available from, e.g., Beckman Instruments, Zymark Inc. or the like.

Alternatively, or additionally, fluid handling systems can include sample material sources (vessels or reservoirs) integrated into the overall packaging of the sensor device, where the sources are fluidly connected to the sensing surface via one or more fluidic channels integrated into that packaging. In such cases, in addition to the fluidic channel network, the fluid handling system will also typically include the actuation systems for controllably moving fluids from their respective sources to the sensing surface, or surfaces, in the case of multiplexed sensor devices. Examples of such integrated fluid handling systems are generally described in the patent literature from Caliper Technologies Corp. In some cases, fluidic elements can be fabricated into the sensor device, e.g., by incorporating a channel or conduit containing layer over the insulator and semiconductor layer that comprises the sensing element. In particular, a plate having a series of channels defined in it, as grooves, can be mated with and bonded to, a plate or wafer having a sensor element (and optionally additional circuitry) defined thereon, such that the fluidic elements, e.g., channels or grooves, are disposed, at least partially. Alternatively, a pipettor system 906 may be integrated into the device that houses the sensor element, and thus integrates the accession of samples with the analysis of samples.

In addition to fluid handling systems, the overall systems of the invention typically include data processing capability to enable the programmed operation of the sensor devices and systems, and to receive, store and provide useful analysis and display of the data that is obtained. Typically, such processing capability is at least partially provided through an external computer 914 that is coupled (via electrical cable 912) to the power supply and monitoring portion of the system 100. In addition to that portion of the processing capability provided by the computer, additional processor(s) may be integrated into the sensor system itself. By way of example, instruction sets that are routinely run during set up, operation and shut down are conveniently integrated into a sensor system. These typically include power adjustments, diagnostic operations, and the like. As used hereafter in the description, the processing capabilities of the overall system, whether internally or externally integrated with the overall system are referred to as the computer. This reference is merely for ease of discussion and does not alter the basic description of internal and external processing capability, provided above.

Typically, the computer will include appropriate programming to automate operation of the sensor system 100, as well as its ancillary components, e.g., the fluid handling system 906, via electrical cable 916. This includes instructing the power supply in applying, regulating and/or modulating the current applied to the sensor, and instructing the fluid handling system, to the extent necessary, to deliver sample materials to the sensing surface of the sensor. The computer also will include programming that enables the receipt and storage of conductance data received from the sensor system 100 in response to the application of sample material with the sensing surface. In addition, the computer will also typically be programmed to provide meaningful analysis of the conductance data. For example, the computer may include programming that interprets conductance data received from the sensor and provides the user with a determination of the presence and optionally, the concentration of an analyte of interest. Such programming may include access to a look-up table of known conductance values for different analyte concentrations. Alternatively, the programming will compare conductance values against conductance values for a standard or control sample or range of control samples, and extrapolate or interpolate the concentration of the analyte of interest in the sample.

In a simple exemplary operation, and with reference to FIG. 9, a computer, at a user's initiation instructs the fluid handling system 906 to obtain a fluid sample 904 from source 902. In the case shown, this is accomplished by placing a pipette tip 908 into contact with the fluid sample 904 and drawing a portion of the fluid into the tip. The pipette tip is then translocated to a position over the sensor reservoir 910, at which point the computer 914 instructs the pipettor to expel the fluid into the reservoir 910. As noted previously, although the fluid handling system is generally referred to as a pipetting system, it will be understood that integrated fluidic networks that may include pipettors that are also integrated into the system can also function as the fluid handling system (see, e.g., AMS90SE pipetting analysis systems and chips available from Caliper Technologies Corp). Similarly, although illustrated as a simple reservoir, the sensing reservoir 910 optionally includes a variety of fluidic components, including inlet ports and channel manifolds for delivering sample material to the sensing surface, or to multiple different or duplicate sensing surfaces. The computer then instructs the sensor system 100 to measure the response of the sensor to the sample material. Based upon appropriate software, the computer analyzes the information and provides a user understandable display of the results of the analysis.

V. Exemplary Fabrication Process for IC Nanowire for Nanosensor Applications

The following process is an exemplary proposed fabrication process for planar nanowires for use in nanosensor applications according to certain aspects of the present invention, starting with a silicon on insulator, SOI, wafer as the substrate.

The substrate is processed using standard photoresist application procedures, e.g., as described in the Choudhury, Handbook of Microlithography, Micromachining, and Microfabrication Vols. 1 and 2, (SPIE Press, 1997), which is incorporated herein by reference in its entirety for all purposes. Such processing might include initial preparation steps including singing the wafer to remove unwanted absorbed gases and water on the substrate surface, and treatment of the substrate with photoresist adhesion promoters, e.g. hexamethyldisilazane (HMDS).

A photoresist layer is then spin coated onto the silicon side of the substrate wafer. One exemplary e-beam resist is polymethylmethacrylate (PMMA) which is generally available in resist formulations, e.g., from Microchem Inc. (Newton Mass.). While described in terms of using a PMMA resist, it will be appreciated that any number of different resists are available that could be used in the process, including, e.g., ZEP 520-12 (Zeon Co., Japan) and HSQ (Dow-Corning, Michigan). To write sub 100 nanometer features, e-beam lithography can be used, which has the capability of imaging down to ˜10 nm. Alternatively, conventional lithography may be used where sub 100 nm widths are not required, or additional etching processes are employed to yield sub 100 nm features, e.g., over etching or isotropic etching processes. Typically, a single wafer will have a number of discrete devices fabricated therefrom, e.g., a number of separate nanowire sensor devices will be simultaneously fabricated onto a single wafer.

Since e-beam lithography can be relatively slow by comparison to photolithography, e-beam lithography is generally used to image small nanowires with contact pads, while more conventional lithography will be used to image the areas around the small nanowires and contact pads. Other lithography techniques such as interference lithography (small feature), projection lithography (large feature) or any combination of the small feature and large feature generating lithography types can be used in conjunction with each other. After the wafer has been exposed using the e-beam lithography system, additional exposure steps may be used, e.g., to expose areas not immediately surrounding the nanowire element.

Following the exposure step, the resist is developed in accordance with the procedures optimized for the particular resist, e.g., as recommended by the manufacturer or as set forth in Chaudhury, above. To transfer the developed features in the PMMA photoresist to silicon, the wafer containing the developed photoresist is subjected to the etching process step. As noted previously, this is preferably carried out by plasma or RIE etching processes. However, as also noted previously, wet chemical etching steps may also be involved, for certain embodiments.

In the case of plasma etching, the wafer is first briefly exposed to a fluorine plasma generated from CF₄ to fluorinate the top layer of photoresist, thereby increasing the photoresist's etch resistance to chlorine. The wafer is then exposed to an etch plasma, e.g., a chlorine and argon plasma resulting from the decomposition of BCl₃ and Ar, to etch away the silicon and leave a nanowire with contact pads remaining on the silicon oxide surface. Any number of chemical compounds containing fluorine, e.g., C₄F₈, SF₆, CHF₃ and NF₃ can be used in place CF₄. Similarly any number of chlorine compounds, e.g., Cl₂ and other chlorine containing compounds can be used in place of BCl₃. One skilled in the art can easily tune a chlorine plasma to selectively only etch silicon and not etch the underlying oxide in the SOI wafer. Typically, following the etch step, the wafer is passivated either in the cluster tool or is dipped in water and dried in a spin rinse drier (SRD). This process converts the surface bonded chlorine to hydrochloric acid and removes the chlorine from the wafer surface where it could cause problems in subsequent processing.

Following the definition of the nanowire element, additional features may also be fabricated onto the substrate, e.g., patterning electrodes onto the substrate to provide interface components for the nanowire element and any external instrumentation used in its operation. For example, following treatment of the wafer with an organic solvent to remove any residual PMMA left on the surface of the wafer, the wafer may be spin coated with a lift-off resist for subsequent metallization (positive or negative, depending upon the mask strategy). The lift-off photoresist is baked, exposed and developed to leave small openings in the resist layer over the contact pads for the nanowire element, which openings also extend to a desired point in the device structure for interfacing with external electrical contacts, e.g., edge connectors, pins, or the like. A thin layer of metal, e.g., aluminum or aluminum/silicon, is sputtered over the openings at a desired thickness, e.g., 250 Angstroms. Any number of other metals could be used for this purpose provided they are capable of forming ohmic contacts with the silicon layer underneath. The lift-off photoresist is removed with the appropriate solvent such as acetone, lifting off the aluminum that had deposited on the side of the troughs and on top of the lift-off photoresist.

In at least one aspect, aluminum alloy is employed in forming ohmic contacts with the nanowire element of the device. In particular, in many cases, gold and/or indium are used to form ohmic contacts for nanowires. These metals are selected because they diffuse rapidly into silicon at relatively low temperatures, providing good ohmic contact with relatively little effort and avoiding extreme conditions for processing. While such contacts are quite acceptable from an operational standpoint, they provide a number of difficulties from a manufacturing standpoint, including their incompatibility with most fabrication operations, e.g., contamination, and expense. While use of aluminum contacts addresses issues of cost and contamination, as it is commonly used in integrated circuit fabrication, it can pose problems for contacting nanowire elements. In particular, in annealing an aluminum contact, a small amount of silicon dissolves into the aluminum. Loss of even a small amount of the silicon in a nanowire element could result in a substantial weakening or even breakage of a nanoscale wire element. See, above. As a result, alloys of aluminum and silicon, e.g., a 1% silicon containing alloy is used. The presence of silicon within the alloy reduces the amount of silicon that can be dissolved into the contact, thereby reducing any losses associated therewith. In addition, the alloy may include copper as well as silicon, in order to improve resistance of the contact to electromigration. Typically, the amount of silicon and or copper in the alloy may be varied depending upon the desired properties of the contact. Such variations would be readily selected by one of ordinary skill in the art with only modest, if any, experimentation. While described in terms of the planar processed wire elements described herein, such contacts are also useful in providing electrical contacts to free standing nanowire elements, and perhaps even to provide greater benefits to such processes.

In many cases, it will be desirable to utilize backside contacts for any wire element, e.g., ohmic contacts that are coupled to electrical pins or conductive paths that pass through the substrate itself. In particular, for sensor applications, the wire element is typically exposed to fluids, e.g., aqueous solutions. As a result, it becomes important to effectively seal the ohmic contacts for the sensor from the fluid contact region of the sensor to avoid shorting out of the sensor. See, above. While epoxy or other polymer layers may be used to provide this sealing function, such layers tend to leak over time, and are thus not always suitable options, e.g., for long term or repeated use of sensor devices. As such, integrating the electrical contacts through the back of the substrate, and thus blocking them from the fluid via the silicon, or other semiconductor layer, provides a virtually permanent seal to these contacts. A nanowire based device including such backside contact elements is shown in FIG. 10 from a side and top view. As shown, the device 1000 comprises a planar structure as described herein, and is fabricated from a substrate that includes a base semiconductor substrate 1002, an insulator layer 1004 and a device layer 1006 of semiconductor material. Vias 1008 or passages are fabricated through the overall substrate, e.g., by etching, where the vias will be proximal to the regions of the nanowire element 1010 to which ohmic contact is desired. A conductive element 1012, typically a metal, e.g., gold, is deposited into the vias 1008, which deposition is optionally aided through the inclusion of seeding/adhesion layer 1014 in the vias 1008. A polysilicon base 1016 or cap is then provided on the device layer 1006 in order to provide the connection between the metal conductive path or element 1012 and the nanowire element 1010 defined on the surface of the substrate.

Although described in terms of a nanowire based sensor that is fabricated in a planar orientation, it will be appreciated that such backside contacts may be employed to make ohmic contact to a variety of different nanostructures, e.g., nanowires, including nanowires that are fabricated as free standing structures and subsequently are positioned and secured to a base substrate, nanowires that extend vertically from a substrate or surface of a substrate, etc.

A variety of methods may be employed in fabricating backside contacts for nanowire based devices that employ well known semiconductor fabrication processes, e.g., in etching vias, depositing metals, and polysilicon. For example, in some cases, it may be desirable to fabricate the backside contacts prior to defining the nanowire element on a substrate, whether through lithography or by attaching a free standing wire. Alternatively, however, it may be desirable to first define the wire element on the substrate surface, and then provide the backside contact. Similarly, one may deposit the polysilicon base before or after the metal backside contact element is fabricated. The order may depend upon the compatibility of the metal with the polysilicon deposition process. In particular, polysilicon deposition is generally carried out at temperatures of between about 600° C. and 800° C. As such, for metals that are subject to any degradation, e.g., melting or other reactions, at these temperatures, it will generally be desired to deposit those metals after polysilicon deposition. In contrast, other metals that are reasonably resilient to these temperatures may be deposited before or after the polysilicon deposition.

Additional processing steps, e.g., for depositing nitride layers or additional metal layers can be carried out in accordance with these processes and/or those set forth in Chaudhury, supra. Following the processing, the wafer is diced into individual devices, e.g., using a scribe and break, or sawing method, and each device is packaged for use.

### EXAMPLE 1

### Charge Sensitivity of SOI Nanowire Elements

A number of nanowire elements, were fabricated from an SOI substrate wafer having a device layer thickness of approximately 50 nm, using e-beam lithography to define wires or gate portions having different widths of 200, 500, 800, 1100, 1400 and 1700 nm. The gate regions each spanned two substantially wider regions for making electrical contact. The 800 and 1100 nm wide wires were then used for additional testing. The nanowire elements were unmodified but for a native oxide layer. Connecting wires were bonded to the two wider regions for measuring conductance across the nanowire element as the source and drain electrodes. A glass cylinder was glued over the gate portion of the wire element between the electrical wire connections to permit fluid contact to the wire without shorting out the electrical connections. A reference electrode (AgCl) was placed into the glass cylinder to control the potential of the solution with respect to the nanowire element. A series of different buffers (10 mM with 150 mM salt concentrations were applied to the wire element (pH 4, 5, 7, 8, 9, and 10)).

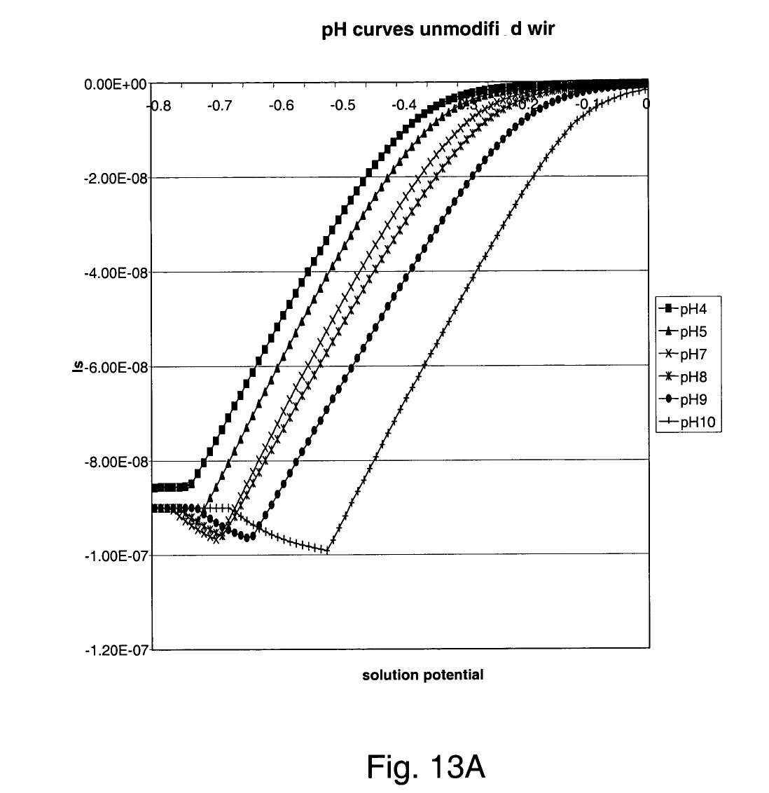

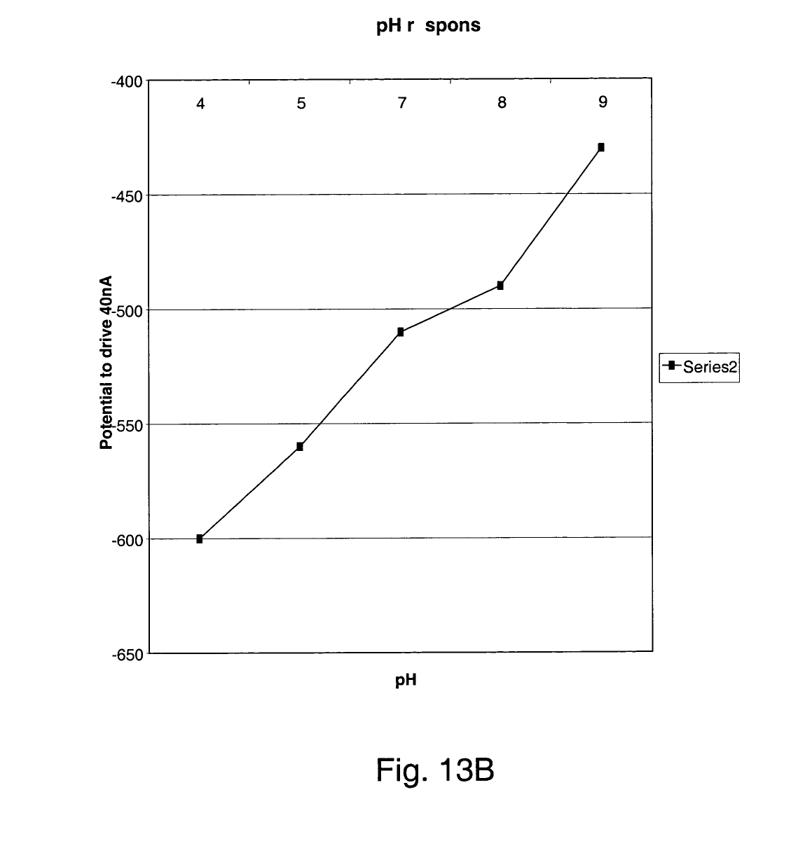

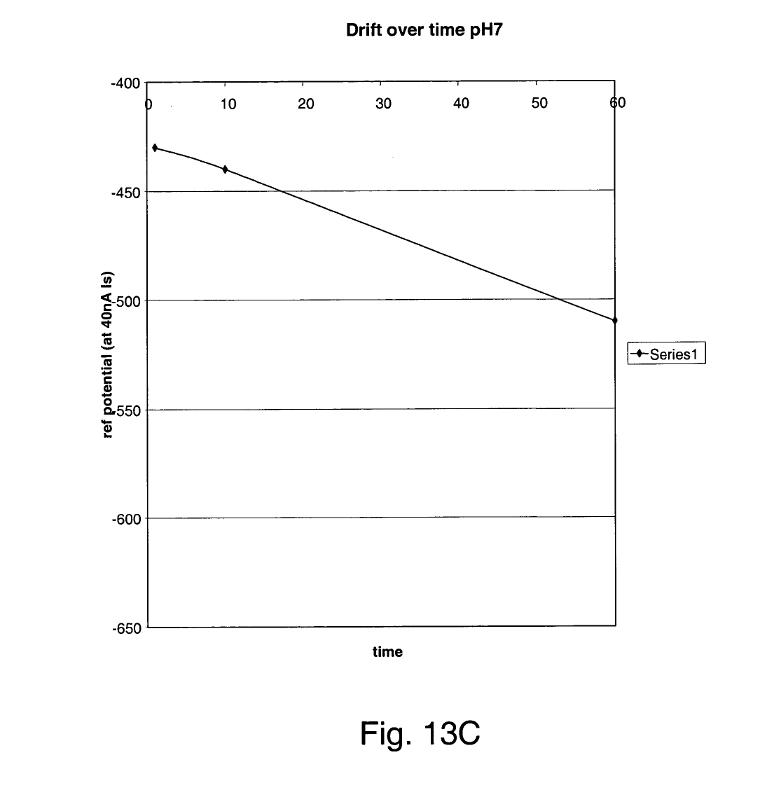

The solution potential was adjusted upwards in 10 mV increments and the current flow through the source electrode (Is) was measured for each different pH. A plot of the potential sweep vs. Is for each pH solution is shown in FIG. 13A. FIG. 13B illustrates a plot of the solution potential required to drive 40 nA of current at each pH. FIG. 13C then shows the signal drift of the wire at pH 7, over time.

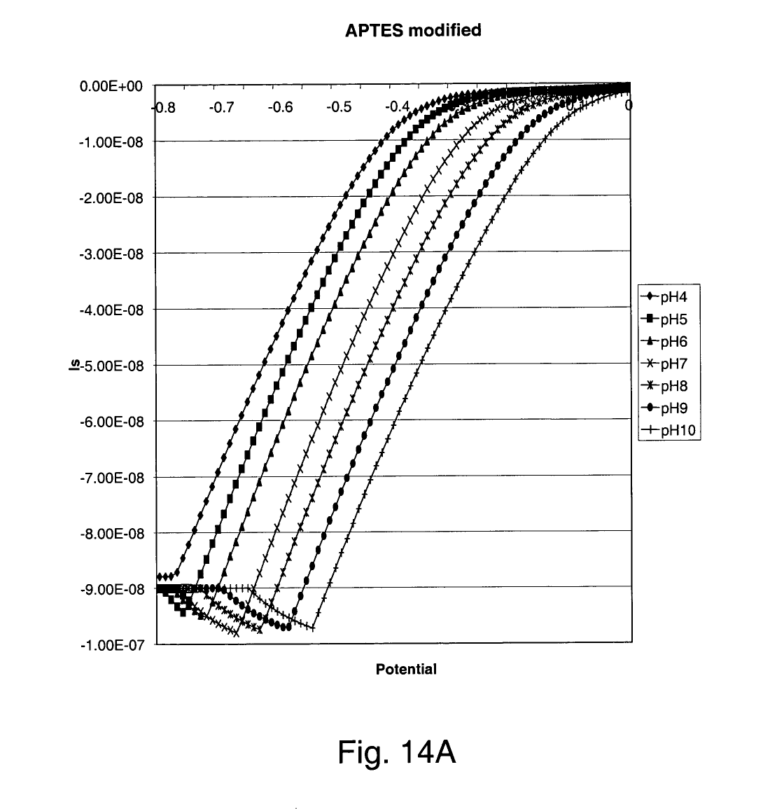

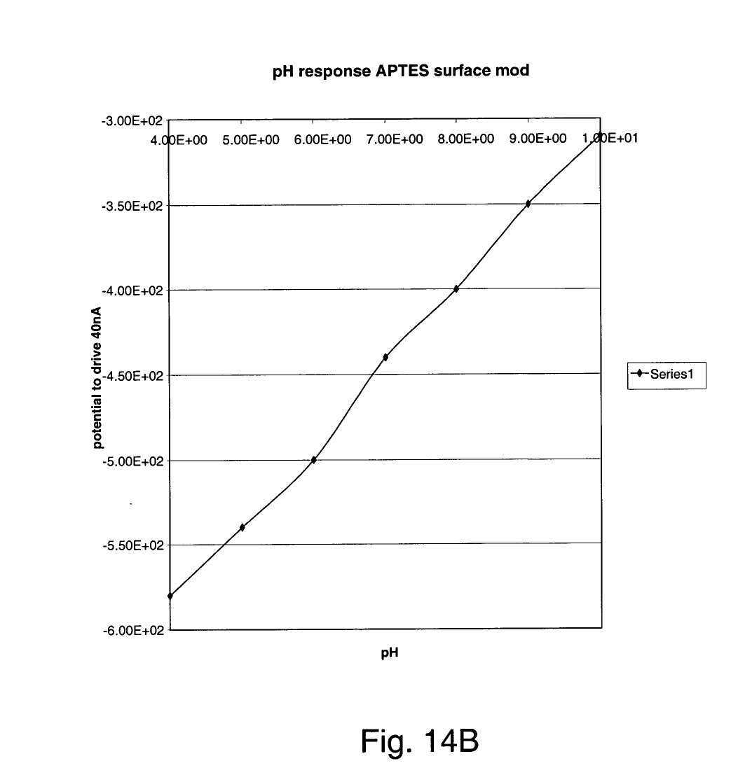

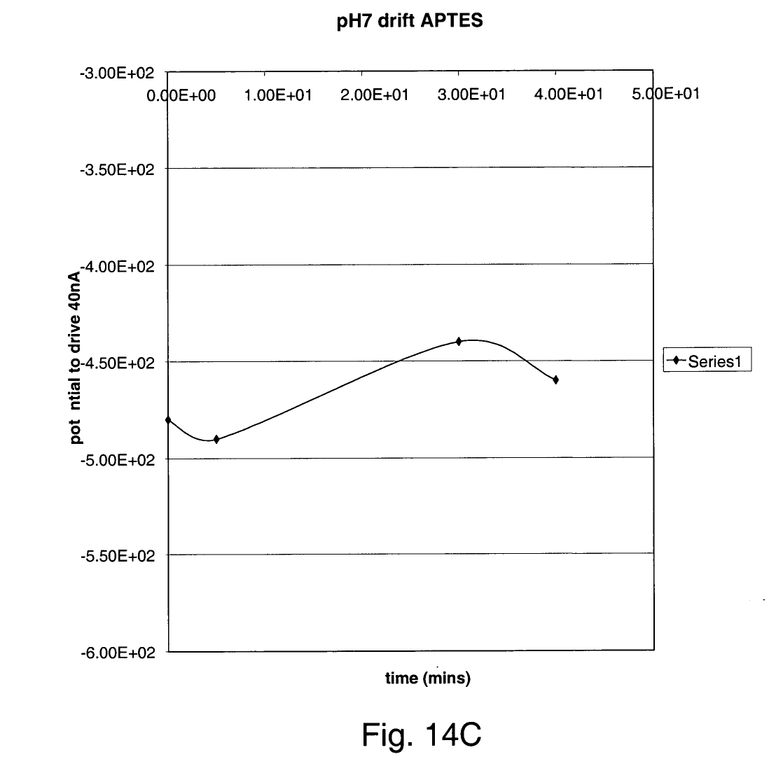

The same processes were performed on wire elements that were modified with APTES. FIGS. 14A, B and C show the same plots as FIGS. 13A, B and C, except using modified wires. As can be seen, the pH response is substantially more linear in modified wires, while the drift appears to have been modestly reduced.

### EXAMPLE 2

### Encapsulation of Metal Traces

SU-8 was used to insulate the Al metal traces for the drain and source electrodes in a sensor array having a gap of 20 μm between the two electrodes. The SU-8 insulator was patterned to leave an opening of 2, 5, 10 or 15 μm on four separate structures to form the fluid channels.

The first test consisted of applying 100 volts (limited to 10 nA) to the solution surrounding the system (i.e., the two electrodes) to measure any leakage. All the devices on 2 chips subjected to this condition, i.e., 8, exhibited very low leakage (˜100 to 200 pA).

Reliability tests indicated very low leakages at the 10 V signal level. Devices were soaked under 1 M NaCl and the leakage characterized to be ˜200 pA. A 2 V potential was applied to a 1 M solution with respect to the two metal traces and left for about 42 hours, at which time the leakage was characterized to be the same as for the initial leakage. The potential was then increased to 10 V for another 20 hours and the leakage did not change. See, FIG. 15.

All publications and patent applications are herein incorporated by reference to the same extent as if each individual publication or patent application was specifically and individually indicated to be incorporated by reference. Although the present invention has been described in some detail by way of illustration and example for purposes of clarity and understanding, it will be apparent that certain changes and modifications may be practiced within the scope of the appended claims.

What is claimed is:

1. A nanosensor, comprising:
a semiconductor element integral to an insulating substrate, and having length and width dimensions parallel to the insulating substrate, and a depth dimension orthogonal to the insulating substrate, the depth dimension being less than 500 nm; and
a sensing surface electrically coupled to the semiconductor element, the sensing surface comprising at least a first functional moiety that is capable of interacting with a first analyte of interest.

2. The nanosensor of claim 1, wherein the depth dimension of the semiconductor element is less than 200 nm.

3. The nanosensor of claim 1, wherein the depth dimension of the semiconductor element is less than 100 nm.

4. The nanosensor of claim 1, wherein the depth dimension of the semiconductor element is less than 50 nm.

5. The nanosensor of claim 1, wherein the depth dimension of the semiconductor element is less 25 nm.

6. The nanosensor of claim 1, wherein the depth dimension of the semiconductor is between about 15 nm and about 100 nm.

7. The nanosensor of claim 1, wherein the semiconductor element comprises silicon, and the insulator comprises silicon dioxide.

8. The nanosensor of claim 1, wherein the first functional moiety comprises a biochemical.

9. The nanosensor of claim 1, wherein the first functional moiety comprises a metal.

10. The nanosensor of claim 1, wherein the first functional moiety comprises a metal oxide.

11. The nanosensor of claim 8, wherein the first functional moiety comprises one member of: a receptor:ligand pair, a binding protein:ligand pair, an antibody:epitope pair, an antibody fragment:epitope pair, a pair of complementary oligonucleotides, or a phosphorylated protein:multivalent metal ion pair.

12. The nanosensor of claim 1, wherein the sensing surface comprises the first functional moiety coupled directly to a surface of the semiconductor element.

13. The nanosensor of claim 12, wherein the first functional moiety is directly coupled to the surface of the semiconductor element via a linker molecule.

14. The nanosensor of claim 1, wherein the sensing surface comprises the first functional moiety associated with a layer disposed over the semiconductor element.

15. The nanosensor of claim 14, wherein the layer disposed over the semiconductor element comprises an insulator layer.

16. The nanosensor of claim 14, wherein the layer disposed over the semiconductor element comprises a metal layer.

17. The nanosensor of claim 16, wherein the metal layer comprises a metal oxide layer.

18. The nanosensor of claim 16, wherein the metal layer is selected from gold, platinum, or tin.

19. The nanosensor of claim 1, wherein the semiconductor element comprises first and second segments, the first and second segments comprising different doping.

20. The nanosensor of claim 1, further comprising at least a first electrical circuit, electrically coupled to the semiconductor element.

21. The nanosensor of claim 20, wherein the at least first electrical circuit comprises a buffering circuit.

22. The nanosensor of claim 20, wherein the at least first electrical circuit comprises a multiplexing circuit, said multiplexing circuit being electrically coupled to at least one additional semiconductor element.

23. The nanosensor of claim 20, wherein the at least first electrical circuit comprises an amplification circuit.

24. The nanosensor of claim 23, wherein the additional semiconductor element is integral to an insulating substrate, has a length and width dimensions parallel to the insulating substrate, and a depth dimension orthogonal to the insulating substrate, the depth dimension being less than 100 nm, and a sensing surface electrically coupled to the semiconductor element, the sensing surface comprising a second functional moiety for interacting with a second analyte of interest.

25. The nanosensor of claim 24, wherein the second functional moiety is different from the first functional moiety.

26. The nanosensor of claim 1, further comprising first and second electrical contacts electrically coupled to different points along the length dimension of the semiconductor element.

27. A nanosensor, comprising:
a semiconductor element having a longitudinal axis, and attached to an insulating substrate such that the longitudinal axis is parallel to the insulating substrate, wherein the semiconductor element comprises a depth dimension orthogonal to the substrate that is less than 500 nm;
first and second electrical contacts in electrical communication with the semiconductor element at first and second different points along the longitudinal axis, respectively; and,
a sensing surface electrically coupled to the semiconductor element, having at least a first functional moiety immobilized thereon, wherein interaction of an analyte of interest with the functional moiety induces a change in an electrical property of the semiconductor element.

28. An array, comprising:
a first nanosensor element comprising a first semiconductor element integral to an insulating substrate, and having a length and width dimensions parallel to the insulating substrate, and a depth dimension orthogonal to the insulating substrate, the depth dimension being less than 500 nm, and a first sensing surface electrically coupled to the semiconductor element, the first sensing surface comprising at least a first functional moiety for interacting with a first analyte of interest; and,
at least a second nanosensor element comprising a second semiconductor element integral to an insulating substrate, and having a length and width dimensions parallel to the insulating substrate, and a depth dimension orthogonal to the insulating substrate, the depth dimension being less than 500 nm, and a sensing surface electrically coupled to the second semiconductor element, the second sensing surface comprising at least a second functional moiety for interacting with a second analyte of interest.

29. The array of claim 28, wherein the depth dimensions of the first and second semiconductor elements are less than 200 nm.

30. The array of claim 28, wherein the depth dimensions of the first and second semiconductor elements are less than 100 nm.

31. The array of claim 28, wherein the first and second nanosensor elements are independently electrically addressable.

32. The array of claim 28, wherein the first and second nanosensor elements are disposed in a single fluid reservoir.

33. The array of claim 28, wherein the first and second nanosensor elements are each electrically coupled to a multiplexing circuit.

34. The array of claim 28, wherein the first and second functional moieties are different.

35. The array of claim 28, wherein the first and second analytes of interest are different.

36. The array of claim 28, wherein the first and second analytes of interest are the same analyte.

37. The array of claim 28, wherein the first and second nanosensor elements are disposed in different fluid reservoirs.

38. The array of claim 28, wherein the first and second functional moieties are the same functional moiety.

39. A method of fabricating a nanosensor, comprising:
providing a semiconductor layer on an insulating substrate, wherein the semiconductor layer is less than 500 nm thick;
defining an elongated structure from the semiconductor layer, the structure having length and width dimensions that are parallel to the insulating substrate, and a depth dimension orthogonal to the insulating substrate that is less than 500 nm; and,
providing a sensing surface electrically coupled to the elongated structure, the sensing surface comprising a functional moiety that interacts with an analyte of interest to induce a change in an electrical property of the elongated structure.

40. The method of claim 39 wherein the step of providing a semiconductor layer on an insulating substrate comprises providing a semiconductor layer that is less than 200 nm thick.

41. The method of claim 39, wherein the step of providing a semiconductor layer on an insulating substrate comprises providing a semiconductor layer that is less than 100 nm thick.

42. The method of claim 39, wherein the depth dimension is substantially equal to the thickness.

43. The method of claim 39, wherein the semiconductor layer on an insulating substrate comprises a semiconductor on insulator substrate.

44. The method of claim 43, wherein the semiconductor on insulator substrate comprises a silicon on insulator (SOI) substrate.

45. The method of claim 44, wherein the SOI substrate comprises a silicon layer on a silicon dioxide layer.

46. The method of claim 43, wherein the semiconductor on an insulator substrate comprises a SiMOX wafer.

47. The method of claim 39, wherein the defining step comprises:
coating the semiconductor layer with a resist;
exposing and developing the resist to produce a pattern in the resist that corresponds to the structure to be defined;
protecting the pattern that corresponds to the structure to be defined; and,
removing the semiconductor layer that does not correspond to the structure to be defined, thereby defining the structure.

48. The method of claim 47, wherein the exposing step comprises irradiating defined portions of the resist with an electron beam.

49. The method of claim 47, wherein the exposing step comprises irradiating defined portions of the resist with light.

50. An analytical system, comprising:
a nanosensor, comprising:
a semiconductor element integral to an insulating substrate, and having a length and width dimensions parallel to the insulating substrate, and a depth dimension orthogonal to the insulating substrate, the depth dimension being less than 500 nm, and a ratio of the length dimension to the depth dimension being greater than 500;
a sensing surface electrically coupled to the semiconductor element, the sensing surface comprising a functional moiety capable of interacting with an analyte of interest; and,
a detector electrically coupled to the nanosensor for measuring conductance of the semiconductor element.

51. The system of claim 50, wherein the depth dimension of the semiconductor element is less than 200 nm.

52. The system of claim 50, wherein the depth dimension of the semiconductor element is less than 100 nm.

53. The system of claim 50, further comprising a fluid containing vessel, the sensing surface of the nanosensor being at least partially disposed within the fluid vessel.

54. The system of claim 53, wherein the fluid containing vessel comprises a fluidic conduit.

55. The system of claim 53, wherein the fluid containing vessel comprises a microfluidic channel.

56. The system of claim 53, wherein the fluid containing vessel comprises a well in a multiwell plate.

57. The system of claim 50, further comprising a computer operably coupled to the detector, the computer being operably programmed to receive and store conductance data from the detector.

58. The system of claim 50, further comprising a fluid handling system fluidly connected to the nanosensor for directing fluid samples into contact with the sensing surface of the nanosensor.

59. A method of analyzing a sample material, comprising:
providing a nanosensor comprising:
a semiconductor element integral to an insulating substrate, and having a length and width dimensions parallel to the insulating substrate, and a depth dimension orthogonal to the insulating substrate, the depth dimension being less than 500 nm;
a sensing surface electrically coupled to the semiconductor element, the sensing surface comprising a functional moiety capable of interacting with an analyte of interest; and,
contacting a sample material with the sensing surface of the nanosensor; and
determining a concentration of the analyte of interest in the sample material.

60. The method of claim 60, wherein the depth dimension of the semiconductor element is less than 200 nm.

61. The method of claim 60, wherein the depth dimension of the semiconductor element is less than 100 nm.

62. The method of claim 60, wherein the determining step comprises measuring a conductance of the semiconductor element, and correlating the conductance to a concentration of the analyte of interest.

63. The method of claim 60, wherein the contacting step comprises immersing the sensing surface in the sample material.

64. The method of claim 60, wherein the contacting step comprises flowing the sample material over the sensing surface.
